SEQUENCE LISTING (11 sequences)

SEQ ID NO: 1 - protein, 339 aa, Homo sapiens
MAAAAAAALESWQAAAPRKRRSAARRPRRREAAPRGREAAPRGREAAPRGPEAEFESDSGVVLRRIWEAEKDLFISDFWSSALETINRCLTKHLEQLKAPVGTLSDIFGNLHLDSLPEESDVATDSIPREILVTGTCHLKCVCYGIGNFATCIVARNQLTFLLLLLEKCQIPRSHCWVYDPLFSQLEIEVLNTLGVTVLSENEEGKRSIRGEPTIFYMLHCGTALYNNLLWSNWSVDALSKMVIIGNSFKGLEERLLARILQKNYPYIAKILKGLEELEFPQTSQYMDIFNDTSVHWFPVQKLEQLSIDIWEFREEPDYQDCEDLEIIRNKREDPSATD

SEQ ID NO: 2 - DNA, 81 bp, Artificial sequence
  Synthetic primer
aatgatacggcgaccaccgagatctacactctttccctacacgacgctcttccgatcttttgtggaaaggacgaaacaccg

SEQ ID NO: 3 - DNA, 82 bp, Artificial sequence
  Synthetic primer
aatgatacggcgaccaccgagatctacactctttccctacacgacgctcttccgatctatttgtggaaaggacgaaacaccg

SEQ ID NO: 4 - DNA, 83 bp, Artificial sequence
  Synthetic primer
aatgatacggcgaccaccgagatctacactctttccctacacgacgctcttccgatctgatttgtggaaaggacgaaacaccg

SEQ ID NO: 5 - DNA, 84 bp, Artificial sequence
  Synthetic primer
aatgatacggcgaccaccgagatctacactctttccctacacgacgctcttccgatctcgatttgtggaaaggacgaaacaccg

SEQ ID NO: 6 - DNA, 85 bp, Artificial sequence
  Synthetic primer
aatgatacggcgaccaccgagatctacactctttccctacacgacgctcttccgatcttcgatttgtggaaaggacgaaacaccg

SEQ ID NO: 7 - DNA, 86 bp, Artificial sequence
  Synthetic primer
aatgatacggcgaccaccgagatctacactctttccctacacgacgctcttccgatctatcgatttgtggaaaggacgaaacaccg

SEQ ID NO: 8 - DNA, 87 bp, Artificial sequence
  Synthetic primer
aatgatacggcgaccaccgagatctacactctttccctacacgacgctcttccgatctgatcgatttgtggaaaggacgaaacaccg

SEQ ID NO: 9 - DNA, 88 bp, Artificial sequence
  Synthetic primer
aatgatacggcgaccaccgagatctacactctttccctacacgacgctcttccgatctcgatcgatttgtggaaaggacgaaacaccg

SEQ ID NO: 10 - DNA, 25 bp, Artificial sequence
  Synthetic primer
caagcagaagacggcatacgagata

SEQ ID NO: 11 - DNA, 56 bp, Artificial sequence
  Synthetic primer
tgactggagttcagacgtgtgctcttccgatctccgactcggtgccactttttcaa

(19) United States
(12) Patent Application Publication
(10) Pub. No.: US 2022/0227824 A1
(43) Pub. Date: Jul. 21, 2022

(54) USE OF SRRD IN THE INHIBITION OF PROTEIN AGGREGATION

(71) Applicant: THE CHILDREN'S HOSPITAL OF PHILADELPHIA, Philadelphia, PA (US)

(72) Inventors: Ophir H. SHALEM, Philadelphia, PA (US); Katelyn SWEENEY, Philadelphia, PA (US)

(73) Assignee: THE CHILDREN'S HOSPITAL OF PHILADELPHIA, Philadelphia, PA (US)

(21) Appl. No.: 17/596,622

(22) PCT Filed: Jun. 23, 2020
(86) PCT No.: PCT/US2020/039057

§ 371 Date: Dec. 14, 2021

Related U.S. Application Data
(60) Provisional application No. 62/865,604, filed on Jun. 24, 2019.

(51) Int. Cl. C07K 14/47; C12N 15/86; A61P 25/16; A61P 25/28

(52) U.S. Cl. CPC C07K 14/4702; C12N 15/86; C12N 2750/14143; A61P 25/28; A61P 25/16

(57) ABSTRACT

The present disclosure is directed to the use of SRR1 domain-containing protein (SRRD) or active fragment thereof to reduce protein aggregation, such as that occurring in neurodegenerative diseases.

### PRIORITY CLAIM

This application claims benefit of priority to U.S. Provisional Application Ser. No. 62/865,604, filed Jun. 24, 2019, the entire contents of which are hereby incorporated by reference.

### STATEMENT OF FEDERAL GOVERNMENT GRANT SUPPORT

This invention was made with government support under grant no. 1R03NS111447-01 from the National Institutes of Health. The government has certain rights in the invention.

### BACKGROUND

### 1. Field of the Disclosure

The present disclosure relates generally to the fields of medicine, cell biology, protein chemistry and neurobiology. More particular, the disclosure relates to the use of SRR1 domain-containing protein (SRRD) or active fragment thereof that can reduce protein aggregation, such as that occurring in neurodegenerative diseases.

### 2. Background

Protein aggregation is the underlying cause of a number of neurodegenerative diseases. Such diseases are inherently challenging to treat, which puts a great emphasis on the development new therapeutic techniques. Protein aggregation is a highly complex process that, when not properly controlled, can result in formation of a variety of different aggregates that impair the function of the tissue in which they occur, such as the brain. Indeed, some aggregates are highly cytotoxic.

Therapies are under examination that target different steps in the production and processing of proteins implicated in neurodegenerative disease, including synthesis, chaperone-assisted folding and trafficking, and degradation via the proteasome and autophagy pathways. Other therapies (e.g., mTOR inhibitors and activators of the heat shock response) can serve to rebalance the proteostatic network. While there continues to be an increasing understanding regarding the structure, mechanism of formation, and physiological effects of aggregates, the lack of a complete understanding of the disease development, proper targets and their mechanism of action leaves an urgent need for improved therapies to address protein aggregation-related diseases.

### SUMMARY

Thus, in accordance with the present disclosure, there is provided a method of reducing protein aggregation in a cell comprising providing to a cell SRR1 domain-containing protein (SRRD) or active fragment thereof. Providing may comprise contacting said cell with SRRD or active fragment thereof or contacting said cell with an expression construct encoding SRRD or active fragment thereof, such as a viral expression construct (e.g., such as an adeno- associated virus construct) or a non-viral expression construct. The SRRD or active fragment thereof may further comprise a cell permeability peptide. The SRRD or active fragment thereof or expression construct encoding SSRD or active fragment thereof may be contacted with said cell in a liposome or nanoparticle.

The cell may be a eukaryotic cell, such as a mammalian cell, such as a human cell, or a yeast cell. The SRRD or active fragment thereof may be provided to said cell more than once. The method may further comprise assessing protein aggregation in said cell prior to and/or after said providing, and/or may further comprise providing to said cell a second anti-protein aggregation treatment. The SRRD or active fragment thereof may be provided at a level higher than normally expressed in said cell. The cell may be a mammalian cell and is located in a living mammal.

In another embodiment, there is provided a method of treating a subject suffering from a protein aggregation disease or disorder comprising providing to said subject SRR1 domain- containing protein (SRRD) or active fragment thereof. Providing may comprise administering

SRRD or active fragment thereof or administering an expression construct encoding SRRD or active fragment thereof, such as a viral expression construct (e.g., such as an adeno-associated virus construct) or a non-viral expression construct. The SRRD or active fragment thereof may further comprise a cell permeability peptide. The SRRD or active fragment thereof or expression construct encoding SSRD or active fragment thereof may be administered as part of a liposome or nanoparticle.

The subject may be a mammal, such as a human or a non-human mammal. The SRRD or active fragment thereof may be provided more than once. The method may further comprise assessing protein aggregation in a cell of said subject prior to and/or after said providing, and/or may further comprise providing to said subject a second anti-protein aggregation treatment.

The SRRD or active fragment thereof may be provided at a level higher than normally expressed in a cell of said subject. The protein aggregation disorder or disease may be Alzheimer's Disease, Parkinson's Disease, amyotrophic lateral sclerosis, or frontotemporal dementia. The SSRD or active fragment thereof or expression construct encoding SRRD or active fragment thereof may be administered directly into the central nervous system, such as into the brain, or other interventions that are aimed at elevating endogenous levels of SRRD or active fragment thereof. The SSRD or active fragment thereof or expression construct encoding SRRD or active fragment thereof may be administered systemically, such as intravenously, intra-arterially, orally or subcutaneously.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description below.

FIGS. 1A-D. Pulse Shape Analysis enables the separation of cells with and without TDP-43 aggregates. (FIG. 1A) Cells analyzed using flow cytometry, the plot compares fluorescence height vs width (Pulse Shape Analysis). Analyzed cells are expressing a cytoplasm localized TDP-43 transgene that forms aggregates. A distinct population of cells with aggregates can be observed on the FACS plot. (FIG. 1B) Similar to FIG. 1A only that the cells are expressing a cytoplasm localized TDP-43 transgene that does not form aggregates (due to a different location of the fusion) used as a negative control. (FIGS. 1C-D) Validation of the Pulse Shape approach using microscopy, cells sorted from the non-aggregating (FIG. 1C) or aggregating population (FIG. 1D) display a consistent phenotype when observed under the microscope.

FIG. 2. Illustration of screen experimental outline: Cells stably expressing Cas9 are transduced with a genome-wide library containing 80,000 sgRNAs. Cells are transduced at 1000x coverage (cells/sgRNA). Cells are then selected with puromycin for sgRNA expression and cultured for 10 days to maximize knockout efficiency. At this point the TDP-43 aggregating gene reporter is transiently transfected to cells. After 2 days cells are fixed and sorted to separate aggregating from non-aggerating cells. Sorted cell samples are processed for gDNA extraction and sequencing library preparation.

FIG. 3. Validation of SRRD effect using over expression vector: Cells are transfected with either TDP-43 aggregating plasmid alone or together with a plasmid over expressing SRRD. Percentage of cells with aggregates is then quantified using Pu1SA. A significant reduction in aggregating cells is observed in all three days measured.

FIG. 4. SRRD over expression is protective against TDP-43, FUS and alpha synuclein in a yeast toxicity model: Blue lines in the three panels show yeast growth curves when expressing one of the three toxic proteins TDP-43, FUS and alpha synuclein on the left, middle and right panels respectively. The green lines show co-expression of the toxic protein together with SRRD, a protective effect is observed in all three proteins. The red line on the left panel shows co-expression of SRRD with HSP104, a yeast dissaggregase that has a well-documented protective effect in the yeast model, this is used here as a positive control.

FIG. 5. Expression of only the N-terminus of SRRD increases the reduction in aggregation. Panels show Pu1SA analysis of TDP-43 aggregation co-expressed with different versions of SRRD. From left to right: only TDP-43, TDP-43 +full length SRRD, TDP-43 with N-terminus of SRRD, TDP-43 with SRRD lacking the N-terminus. These panels show that the effect of SRRD is dependent on the N-terminal part of the proteins (last panel on the right) and that expressing only that part of the protein results in a stronger effect.

### DESCRIPTION OF ILLUSTRATIVE EMBODIMENTS

Protein aggregation is a hallmark of almost all neurodegenerative diseases including Alzheimer's Disease (AD), Parkinson's Disease (PD), ALS and others. The inventors recently performed a genome-wide knockout screen using a whole genome CRISPR library in human cell lines to identify proteins that when inactivated affect the propensity of a mutant TDP-43 protein to aggregate. TDP-43 aggregation is observed in ALS, FTD and specific types of Alzheimer's Disease. As one of the strongest gene hits, they identified a protein called SRRD and found that SRRD reduces protein aggregation and toxicity. In addition, overexpression of SRRD was also protective against other unfolded proteins in yeast including Alpha Synuclein. The inventors therefore propose that treatment of the brains of patients with AD, PD, ALS or FTD with SRRD or active fragment thereof will have a protective effect and will delay disease progression.

These and other aspects of the disclosure are described in detail below.

### I. SRRD

SRR1 domain-containing protein (SRRD) may be involved in a circadian clock input pathway and has also been identified as having a role heme synthesis, with the expression of SRRD being dependent on heme biosynthesis. SRRD itself increases the production of heme. SRRD is expressed under circadian rhythms, and influences the expression of clock genes including PER2, BMAL1, and CLOCK. The knockout of SRRD arrests the growth of cells, indicating that SRRD plays roles in heme-regulated circadian rhythms and cell proliferation.

SRRD is located at 22q12.1 of the human genome. It is found in the cytoplasm, has a molecular weight of 38,572 Da and has a predicted pI of 5.31. The amino acid sequence for SRRD is shown below:

(SEQ ID NO: 1)
MAAAAAAALESWQAAAPRKRRSAARRPRRREAAPRGREAAPRGREAAPRG
PEAEFESDSGVVLRRIWEAEKDLFISDFWSSALETINRCLTKHLEQLKAP
VGTLSDIFGNLHLDSLPEESDVATDSIPREILVTGTCHLKCVCYGIGNFA
TCIVARNQLTFLLLLLEKCQIPRSHCWVYDPLFSQLEIEVLNTLGVTVLS
ENEEGKRSIRGEPTIFYMLHCGTALYNNLLWSNWSVDALSKMVIIGNSFK
GLEERLLARILQKNYPYIAKILKGLEELEFPQTSQYMDIFNDTSVHWFPV
QKLEQLSIDIWEFREEPDYQDCEDLEIIRNKREDPSATD

Also contemplated are active fragments of SRRD, including a fragment consisting of the first 50 residues of SEQ ID NO: 1 (underlined above).

### II. Protein Aggregation and Related Diseases

### A. Aggregation

Protein aggregation is a biological phenomenon in which mis-folded proteins aggregate (i.e., accumulate and clump together) either intra- or extracellularly. These protein aggregates are often correlated with diseases. In fact, protein aggregates have been implicated in a wide variety of disease known as amyloidoses, including ALS, Alzheimer's, Parkinson's and prion disease.

After synthesis, proteins typically fold into a particular three-dimensional conformation that is the most thermodynamically favorable: their native state. This folding process is driven by the hydrophobic effect: a tendency for hydrophobic (water-fearing) portions of the protein to shield themselves from the hydrophilic (water-loving) environment of the cell by burying into the interior of the protein. Thus, the exterior of a protein is typically hydrophilic, whereas the interior is typically hydrophobic.

Protein structures are stabilized by non-covalent interactions and disulfide bonds between two cysteine residues. The non-covalent interactions include ionic interactions and weak van der waals interactions. Ionic interactions form between an anion and a cation and form salt bridges that help stabilize the protein. Van der waals interactions include nonpolar interactions (i.e., London dispersion forces) and polar interactions (i.e., hydrogen bonds, dipole-dipole bond). These play an important role in a protein's secondary structure, such as forming an alpha helix or a beta sheet, and tertiary structure. Interactions between amino acid residues in a specific protein are very important in that protein's final structure.

When there are changes in the non-covalent interactions, as may happen with a change in the amino acid sequence, the protein is susceptible to misfolding or unfolding. In these cases, if the cell does not assist the protein in re-folding, or degrade the unfolded protein, the unfolded/misfolded protein may aggregate, in which the exposed hydrophobic portions of the protein may interact with the exposed hydrophobic patches of other proteins. There are three main types of protein aggregates that may form: amorphous aggregates, oligomers, and amyloid fibrils. Protein aggregation can occur due to a variety of causes. There are four classes that these causes can be categorized into, which are detailed below.

Mutations that occur in the DNA sequence may or may not affect the amino acid sequence of the protein. When the sequence is affected, a different amino acid may change the interactions between the side chains that affect the folding of the protein. This can lead to exposed hydrophobic regions of the protein that aggregate with the same misfolded/unfolded protein or a different protein.

In addition to mutations in the affected proteins themselves, protein aggregation could also be caused indirectly through mutations in proteins in regulatory pathways such as the refolding pathway (molecular chaperones) or the ubiquitin-proteasome pathway (ubiquitin ligases). Chaperones help with protein refolding by providing a safe environment for the protein to fold. Ubiquitin ligases target proteins for degradation through ubiquitin modification.

Protein aggregation can be caused by problems that occur during transcription or translation. During transcription, DNA is copied into mRNA, forming a strand of pre-mRNA that undergoes RNA processing to form mRNA. During translation, ribosomes and tRNA help translate the mRNA sequence into an amino acid sequence. If problems arise during either step, making an incorrect mRNA strand and/or an incorrect amino acid sequence, this can cause the protein to misfold, leading to protein aggregation.

Environmental stresses such as extreme temperatures and pH or oxidative stress can also lead to protein aggregation. One such disease is cryoglobulinemia. Extreme temperatures can weaken and destabilize the non-covalent interactions between the amino acid residues. pHs outside of the protein's pH range can change the protonation state of the amino acids, which can increase or decrease the non-covalent interactions. This can also lead to less stable interactions and result in protein unfolding. Oxidative stress can be caused by radicals such as reactive oxygen species (ROS). These unstable radicals can attack the amino acid residues, leading to oxidation of side chains (e.g., aromatic side chains, methionine side chains) and/or cleavage of the polypeptide bonds. This can affect the non-covalent interactions that hold the protein together correctly, which can cause protein destabilization, and may cause the protein to unfold.

Cells have mechanisms that can refold or degrade protein aggregates. However, as cells age these control mechanisms are weakened, and the cell is less able to resolve the aggregates. The hypothesis that protein aggregation is a causative process in aging is testable now since some models of delayed aging are in hand. If the development of protein aggregates was an aging independent process, slowing down aging will show no effect on the rate of proteotoxicity over time. However, if aging is associated with decline in the activity of protective mechanisms against proteotoxicity, the slow aging models would show reduced aggregation and proteotoxicity. To address this problem several toxicity assays have been done in C. elegans. These studies indicated that reducing the activity of insulin/IGF signaling (IIS), a prominent aging regulatory pathway protects from neurodegeneration-linked toxic protein aggregation. The validity of this approach has been tested and confirmed in mammals as reducing the activity of the IGF-1 signaling pathway protected Alzheimer's model mice from the behavioral and biochemical impairments associated with the disease.

Although it has been thought that the mature protein aggregates themselves are toxic, recent evidence suggests that it is in fact immature protein aggregates that are most toxic. The hydrophobic patches of these aggregates can interact with other components of the cell and damage them. The hypotheses are that the toxicity of protein aggregates is related to mechanisms of the sequestration of cellular components, the generation of reactive oxygen species and the binding to specific receptors in the membrane or through the disruption of membranes. A quantitative assay has been used to determine that higher molecular weight species are responsible for the membrane permeation. It is known that protein aggregates in vitro can destabilize artificial phospholipid bilayers, leading to permeabilization of the membrane.

B. Alzheimer's Disease Alzheimer's Disease (AD) is a progressive, neurodegenerative disease characterized by memory loss, language deterioration, impaired visuospatial skills, poor judgment, indifferent attitude, but preserved motor function. AD usually begins after age 65, however, its onset may occur as early as age 40, appearing first as memory decline and, over several years, destroying cognition, personality, and ability to function. Confusion and restlessness may also occur. The type, severity, sequence, and progression of mental changes vary widely. The early symptoms of AD, which include forgetfulness and loss of concentration, can be missed easily because they resemble natural signs of aging. Similar symptoms can also result from fatigue, grief, depression, illness, vision or hearing loss, the use of alcohol or certain medications, or simply the burden of too many details to remember at once.

There is no cure for AD and no way to slow the progression of the disease. For some people in the early or middle stages of the disease, medication such as tacrine may alleviate some cognitive symptoms. Aricept (donepezil) and Exelon (rivastigmine) are reversible acetylcholinesterase inhibitors that are indicated for the treatment of mild to moderate dementia of the Alzheimer's type. Also, some medications may help control behavioral symptoms such as sleeplessness, agitation, wandering, anxiety, and depression. These treatments are aimed at making the patient more comfortable.

AD is a progressive disease. The course of the disease varies from person to person. Some people have the disease only for the last 5 years of life, while others may have it for as many as 20 years. The most common cause of death in AD patients is infection.

The molecular aspect of AD is complicated and not yet fully defined. As stated above, AD is characterized by the formation of amyloid plaques and neurofibrillary tangles in the brain, particularly in the hippocampus which is the center for memory processing. Several molecules contribute to these structures: amyloid β protein (Aβ), presenilin (PS), cholesterol, apolipoprotein E (ApoE), and Tau protein. Of these, Aβ appears to play the central role.

Aβ contains approximately 40 amino acid residues. The 42 and 43 residue forms are much more toxic than the 40-residue form. Aβ is generated from an amyloid precursor protein (APP) by sequential proteolysis. One of the enzymes lacks sequence specificity and thus can generate Aβ of varying (39-43) lengths. The toxic forms of Aβ cause abnormal events such as apoptosis, free radical formation, aggregation and inflammation. Presenilin encodes the protease responsible for cleaving APP into Aβ. There are two forms—PS1 and PS2. Mutations in PS1, causing production of Aβ₄₂, are the typical cause of early onset AD.

Cholesterol-reducing agents have been alleged to have AD-preventative capabilities, although no definitive evidence has linked elevated cholesterol to increased risk of AD. However, the discovery that Aβ contains a sphingolipid binding domain lends further credence to this theory. Similarly, ApoE, which is involved in the redistribution of cholesterol, is now believed to contribute to AD development. As discussed above, individuals having the ApoE4 allele, which exhibits the least degree of cholesterol efflux from neurons, are more likely to develop AD.

Tau protein, associated with microtubules in normal brain, forms paired helical filaments (PHFs) in AD-affected brains which are the primary constituent of neurofibrillary tangles. Recent evidence suggests that Aβ proteins may cause hyperphosphorylation of Tau proteins, leading to disassociation from microtubules and aggregation into PHFs.

### C. Parkinson's Disease

Parkinson's disease (PD) is a long-term degenerative disorder of the central nervous system that mainly affects the motor system. The symptoms generally come on slowly over time. Early in the disease, the most obvious are shaking, rigidity, slowness of movement, and difficulty with walking. Thinking and behavioral problems may also occur. Dementia becomes common in the advanced stages of the disease. Depression and anxiety are also common occurring in more than a third of people with PD. Other symptoms include sensory, sleep, and emotional problems. The main motor symptoms are collectively called “parkinsonism” or a “parkinsonian syndrome.”

The cause of Parkinson's disease is generally unknown but believed to involve both genetic and environmental factors. Those with a family member affected are more likely to get the disease themselves. There is also an increased risk in people exposed to certain pesticides and among those who have had prior head injuries while there is a reduced risk in tobacco smokers and those who drink coffee or tea. The motor symptoms of the disease result from the death of cells in the substantia nigra, a region of the midbrain. This results in not enough dopamine in these areas. The reason for this cell death is poorly understood but involves the build-up of proteins into Lewy bodies in the neurons. Diagnosis of typical cases is mainly based on symptoms, with tests such as neuroimaging being used to rule out other diseases.

There is no cure for Parkinson's disease. Initial treatment is typically with the anti-parkinson medication L-DOPA (levodopa), with dopamine agonists being used once levodopa becomes less effective. As the disease progresses and neurons continue to be lost, these medications become less effective while at the same time they produce a complication marked by involuntary writhing movements. Diet and some forms of rehabilitation have shown some effectiveness at improving symptoms. Surgery to place microelectrodes for deep brain stimulation has been used to reduce motor symptoms in severe cases where drugs are ineffective. Evidence for treatments for the non-movement-related symptoms of PD, such as sleep disturbances and emotional problems, is less strong.

In 2013, PD was present in 53 million people and resulted in about 103,000 deaths globally. Parkinson's disease typically occurs in people over the age of 60, of which about one percent are affected. Males are more often affected than females. When it is seen in people before the age of 40 or 50, it is called young onset PD. The average life expectancy following diagnosis is between 7 and 14 years.

The term parkinsonism is used for a motor syndrome whose main symptoms are tremor at rest, stiffness, slowing of movement and postural instability. Parkinsonian syndromes can be divided into four subtypes, according to their origin (1) primary or idiopathic, (2) secondary or acquired, (3) hereditary parkinsonism, and (4) Parkinson plus syndromes or multiple system degeneration.

Parkinson's disease is the most common form of parkinsonism and is usually defined as “primary” parkinsonism, meaning parkinsonism with no external identifiable cause. In recent years several genes that are directly related to some cases of Parkinson's disease have been discovered. Although this conflicts with the definition of Parkinson's disease as an idiopathic illness, genetic parkinsonism disorders with a similar clinical course to PD are generally included under the Parkinson's disease label. The terms “familial Parkinson's disease” and “sporadic Parkinson's disease” can be used to differentiate genetic from truly idiopathic forms of the disease.

Usually classified as a movement disorder, PD also gives rise to several non-motor types of symptoms such as sensory deficits, cognitive difficulties, and sleep problems. Parkinson plus diseases are primary parkinsonisms which present additional features. They include multiple system atrophy, progressive supranuclear palsy, corticobasal degeneration, and dementia with Lewy bodies. In terms of pathophysiology, PD is considered a synucleiopathy due to an abnormal accumulation of alpha-synuclein protein in the brain in the form of Lewy bodies, as opposed to other diseases such as Alzheimer's disease where the brain accumulates tau protein in the form of neurofibrillary tangles. Nevertheless, there is clinical and pathological overlap between tauopathies and synucleinopathies. The most typical symptom of Alzheimer's disease, dementia, occurs in advanced stages of PD, while it is common to find neurofibrillary tangles in brains affected by PD. Dementia with Lewy bodies (DLB) is another synucleinopathy that has similarities with PD, and especially with the subset of PD cases with dementia. However, the relationship between PD and DLB is complex and still has to be clarified. They may represent parts of a continuum or they may be separate diseases.

### 1. Signs and Symptoms

Parkinson's disease affects movement, producing motor symptoms. Non-motor symptoms, which include autonomic dysfunction, neuropsychiatric problems (mood, cognition, behavior or thought alterations), and sensory and sleep difficulties, are also common. Some of these non-motor symptoms are often present at the time of diagnosis and can precede motor symptoms.

Four motor symptoms are considered cardinal in PD: tremor, rigidity, slowness of movement, and postural instability. Tremor is the most apparent and well-known symptom. It is the most common; though around 30% of individuals with PD do not have tremor at disease onset, most develop it as the disease progresses. It is usually a rest tremor—maximal when the limb is at rest and disappearing with voluntary movement and sleep. It affects to a greater extent the most distal part of the limb and at onset typically appears in only a single arm or leg, becoming bilateral later. Frequency of PD tremor is between 4 and 6 hertz (cycles per second). A feature of tremor is pill-rolling, the tendency of the index finger of the hand to get into contact with the thumb and perform together a circular movement. The term derives from the similarity between the movement of people with PD and the earlier pharmaceutical technique of manually making pills.

Bradykinesia is another characteristic feature of PD and is a slowness in the execution of movement. Performance of sequential and simultaneous movement is hindered. Initial manifestations are problems when performing daily tasks which require fine motor control such as writing, sewing or getting dressed. Clinical evaluation is based on similar tasks such as alternating movements between both hands or between both feet. Bradykinesia is not equal for all movements or times. It is modified by the activity or emotional state of the subject, to the point that some people are barely able to walk yet can still ride a bicycle. Generally, people with PD have less difficulty when some sort of external cue is provided.

Rigidity is stiffness and resistance to limb movement caused by increased muscle tone, an excessive and continuous contraction of muscles. In parkinsonism the rigidity can be uniform (lead-pipe rigidity) or ratchety (cogwheel rigidity). The combination of tremor and increased tone is considered to be at the origin of cogwheel rigidity. Rigidity may be associated with joint pain; such pain being a frequent initial manifestation of the disease. In early stages of Parkinson's disease, rigidity is often asymmetrical and tends to affect the neck and shoulder muscles prior to the muscles of the face and extremities. With the progression of the disease, rigidity typically affects the whole body and reduces the ability to move.

Postural instability is typical in the late stages of the disease, leading to impaired balance and frequent falls, and secondarily to bone fractures. Instability is often absent in the initial stages, especially in younger people. Up to 40% may experience falls and around 10% may have falls weekly, with the number of falls being related to the severity of PD.

Other recognized motor signs and symptoms include gait and posture disturbances such as festination (rapid shuffling steps and a forward-flexed posture when walking), speech and swallowing disturbances including voice disorders, mask-like face expression or small handwriting, although the range of possible motor problems that can appear is large.

Parkinson's disease can cause neuropsychiatric disturbances, which can range from mild to severe. This includes disorders of speech, cognition, mood, behavior, and thought. Cognitive disturbances can occur in the early stages of the disease and sometimes prior to diagnosis and increase in prevalence with duration of the disease. The most common cognitive deficit in affected individuals is executive dysfunction, which can include problems with planning, cognitive flexibility, abstract thinking, rule acquisition, initiating appropriate actions and inhibiting inappropriate actions, working memory, and selecting relevant sensory information. Fluctuations in attention, impaired perception and estimation of time, slowed cognitive processing speed are among other cognitive difficulties. Memory is affected, specifically in recalling learned information. Nevertheless, improvement appears when recall is aided by cues. Visuospatial difficulties are also part of the disease, seen for example when the individual is asked to perform tests of facial recognition and perception of the orientation of drawn lines.

A person with PD has an increased risk of dementia compared to the general population. The prevalence of dementia increases with duration of the disease. Dementia is associated with a reduced quality of life in people with PD and their caregivers, increased mortality, and a higher probability of needing nursing home care.

Behavior and mood alterations are more common in PD without cognitive impairment than in the general population and are usually present in PD with dementia. The most frequent mood difficulties are depression, apathy and anxiety. Establishing the diagnosis of depression is complicated by symptoms that often occur in Parkinson's including dementia, decreased facial expression, decreased movement, a state of indifference, and quiet speech. Impulse control behaviors such as medication overuse and craving, binge eating, hypersexuality, or problem gambling can appear in PD and have been related to the medications used to manage the disease. Psychotic symptoms—hallucinations or delusions—occur in 4% of people with PD, and it is assumed that the main precipitant of psychotic phenomena in Parkinson's disease is dopaminergic excess secondary to treatment; it therefore becomes more common with increasing age and levodopa intake.

In addition to cognitive and motor symptoms, PD can impair other body functions. Sleep problems are a feature of the disease and can be worsened by medications. Symptoms can manifest as daytime drowsiness, disturbances in REM sleep, or insomnia. A systematic review shows that sleep attacks occur in 13.0% of patients with Parkinson's disease on dopaminergic medications.

Alterations in the autonomic nervous system can lead to orthostatic hypotension (low blood pressure upon standing), oily skin and excessive sweating, urinary incontinence and altered sexual function. Constipation and gastric dysmotility can be severe enough to cause discomfort and even endanger health. PD is related to several eye and vision abnormalities such as decreased blink rate, dry eyes, deficient ocular pursuit (eye tracking) and saccadic movements (fast automatic movements of both eyes in the same direction), difficulties in directing gaze upward, and blurred or double vision. Changes in perception may include an impaired sense of smell, sensation of pain and paresthesia (skin tingling and numbness). All of these symptoms can occur years before diagnosis of the disease.

### 2. Causes

Parkinson's disease in most people is idiopathic (having no specific known cause). However, a small proportion of cases can be attributed to known genetic factors. Other factors have been associated with the risk of developing PD, but no causal relationships have been proven.

A number of environmental factors have been associated with an increased risk of Parkinson's, including pesticide exposure, head injuries, and living in the country or farming.

Rural environments and the drinking of well water may be risks, as they are indirect measures of exposure to pesticides. Implicated agents include insecticides, primarily chlorpyrifos and organochlorines and pesticides, such as rotenone or paraquat, and herbicides, such as Agent Orange and ziram. Exposure to heavy metals has been proposed to be a risk factor, through possible accumulation in the substantia nigra, but studies on the issue have been inconclusive.

PD traditionally has been considered a non-genetic disorder; however, around 15% of individuals with PD have a first-degree relative who has the disease. At least 5% of people are now known to have forms of the disease that occur because of a mutation of one of several specific genes.

Mutations in specific genes have been conclusively shown to cause PD. These genes code for alpha-synuclein (SNCA), parkin (PRKN), leucine-rich repeat kinase 2 (LRRK2 or dardarin), PTEN-induced putative kinase 1 (PINK1), DJ-1 and ATP13A2. In most cases, people with these mutations will develop PD. With the exception of LRRK2, however, they account for only a small minority of cases of PD. The most extensively studied PD-related genes are SNCA and LRRK2. Mutations in genes including SNCA, LRRK2 and glucocerebrosidase (GBA) have been found to be risk factors for sporadic PD. Mutations in GBA are known to cause Gaucher's disease. Genome-wide association studies, which search for mutated alleles with low penetrance in sporadic cases, have now yielded many positive results.

The role of the SNCA gene is important in PD, because the alpha-synuclein protein is the main component of Lewy bodies. Missense mutations of the gene (in which a single nucleotide is changed), and duplications and triplications of the locus containing it have been found in different groups with familial PD. Missense mutations are rare. On the other hand, multiplications of the SNCA locus account for around 2% of familial cases. Multiplications have been found in asymptomatic carriers, which indicate that penetrance is incomplete or age-dependent.

The LRRK2 gene (PARKS) encodes a protein called dardarin. The name dardarin was taken from a Basque word for tremor, because this gene was first identified in families from England and the north of Spain. Mutations in LRRK2 are the most common known cause of familial and sporadic PD, accounting for approximately 5% of individuals with a family history of the disease and 3% of sporadic cases. There are many mutations described in LRRK2, however unequivocal proof of causation only exists for a few.

Several Parkinson-related genes are involved in the function of lysosomes, organelles that digest cellular waste products. It has been suggested that some forms of Parkinson may be caused by lysosome dysfunctions that reduce the ability of cells to break down alpha-synuclein.

### 3. Diagnosis

A physician will diagnose Parkinson's disease from the medical history and a neurological examination. There is no medical test that will clearly identify the disease, but brain scans are sometimes used to rule out disorders that could give rise to similar symptoms. People may be given levodopa and resulting relief of motor impairment tends to confirm the diagnosis. The finding of Lewy bodies in the midbrain on autopsy is usually considered proof that the person had Parkinson's disease. The progress of the illness over time may reveal it is not Parkinson's disease, and some authorities recommend that the diagnosis should be periodically reviewed.

Other causes that can secondarily produce a parkinsonian syndrome are Alzheimer's disease, multiple cerebral infarction and drug-induced parkinsonism. Parkinson-plus syndromes such as progressive supranuclear palsy and multiple system atrophy must be ruled out. Anti-Parkinson's medications are typically less effective at controlling symptoms in

Parkinson-plus syndromes. Faster progression rates, early cognitive dysfunction or postural instability, minimal tremor or symmetry at onset may indicate a Parkinson-plus disease rather than PD itself. Genetic forms are usually classified as PD, although the terms “familial Parkinson's disease” and “familial parkinsonism” are used for disease entities with an autosomal dominant or recessive pattern of inheritance.

Medical organizations have created diagnostic criteria to ease and standardize the diagnostic process, especially in the early stages of the disease. The most widely known criteria come from the UK Parkinson's Disease Society Brain Bank and the U.S. National Institute of Neurological Disorders and Stroke. The PD Society Brain Bank criteria require slowness of movement (bradykinesia) plus either rigidity, resting tremor, or postural instability. Other possible causes of these symptoms need to be ruled out. Finally, three or more of the following features are required during onset or evolution: unilateral onset, tremor at rest, progression in time, asymmetry of motor symptoms, response to levodopa for at least five years, clinical course of at least ten years and appearance of dyskinesias induced by the intake of excessive levodopa. Accuracy of diagnostic criteria evaluated at autopsy is 75-90%, with specialists such as neurologists having the highest rates.

Computed tomography (CT) and conventional magnetic resonance imaging (MRI) brain scans of people with PD usually appear normal. These techniques are nevertheless useful to rule out other diseases that can be secondary causes of parkinsonism, such as basal ganglia tumors, vascular pathology and hydrocephalus. A specific technique of MRI, susceptibility weighted imaging has been found to differentiate between patients and subjects without the disease and another technique, diffusion MRI, has been reported to be useful at discriminating between typical and atypical parkinsonism, although its exact diagnostic value is still under investigation. Dopaminergic function in the basal ganglia can be measured with different PET and SPECT radioactive tracers. Examples are ioflupane (¹²³1) (trade name DaTSCAN) and iometopane (Dopascan) for SPECT or fluorodeoxyglucose (¹⁸F) and DTBZ for PET. A pattern of reduced dopaminergic activity in the basal ganglia can aid in diagnosing PD.

### 4. Prevention, Management, Rehabilitation and Palliative Care

Exercise in middle age reduces the risk of Parkinson's disease later in life. Caffeine also appears protective with a greater decrease in risk occurring with a larger intake of caffeinated beverages such as coffee. Although tobacco smoke causes adverse health effects, decreases life expectancy and quality of life, it may reduce the risk of PD by a third when compared to non-smokers. The basis for this effect is not known, but possibilities include an effect of nicotine as a dopamine stimulant. Tobacco smoke contains compounds that act as MAO inhibitors that also might contribute to this effect.

Antioxidants, such as vitamins C and D, have been proposed to protect against the disease, but results of studies have been contradictory and no positive effect has been proven. The results regarding fat and fatty acids have been contradictory, with various studies reporting protective effects, risk-increasing effects or no effects. Also, there have been preliminary indications of a possible protective role of estrogens and anti-inflammatory drugs.

There is no cure for Parkinson's disease, but medications, surgery, and multidisciplinary management can provide relief from the symptoms. The main families of drugs useful for treating motor symptoms are levodopa (usually combined with a dopa decarboxylase inhibitor or COMT inhibitor that does not cross the blood-brain barrier), dopamine agonists and MAO-B inhibitors. The stage of the disease determines which group is most useful. Two stages are usually distinguished: an initial stage in which the individual with PD has already developed some disability for which he needs pharmacological treatment, then a second stage in which an individual develops motor complications related to levodopa usage. Treatment in the initial stage aims for an optimal tradeoff between good symptom control and side-effects resulting from improvement of dopaminergic function. The start of levodopa treatment may be delayed by using other medications such as MAO-B inhibitors and dopamine agonists, in the hope of delaying the onset of dyskinesias. In the second stage the aim is to reduce symptoms while controlling fluctuations of the response to medication. Sudden withdrawals from medication or overuse have to be managed. When medications are not enough to control symptoms, surgery and deep brain stimulation can be of use. In the final stages of the disease, palliative care is provided to improve quality of life.

Levodopa has been the most widely used treatment for over 30 years. L-DOPA is converted into dopamine in the dopaminergic neurons by dopa decarboxylase. Since motor symptoms are produced by a lack of dopamine in the substantia nigra, the administration of L-DOPA temporarily diminishes the motor symptoms.

Only 5-10% of L-DOPA crosses the blood-brain barrier. The remainder is often metabolized to dopamine elsewhere, causing a variety of side effects including nausea, dyskinesias and joint stiffness. Carbidopa and benserazide are peripheral dopa decarboxylase inhibitors, which help to prevent the metabolism of L-DOPA before it reaches the dopaminergic neurons, therefore reducing side effects and increasing bioavailability. They are generally given as combination preparations with levodopa. Existing preparations are carbidopa/levodopa (co-careldopa) and benserazide/levodopa (co-beneldopa). Levodopa has been related to dopamine dysregulation syndrome, which is a compulsive overuse of the medication, and punding. There are slow release versions of levodopa in the form intravenous and intestinal infusions that spread out the effect of the medication. These slow-release levodopa preparations have not shown an increased control of motor symptoms or motor complications when compared to immediate release preparations.

Tolcapone inhibits the COMT enzyme, which degrades dopamine, thereby prolonging the effects of levodopa. It has been used to complement levodopa; however, its usefulness is limited by possible side effects such as liver damage. A similarly effective drug, entacapone, has not been shown to cause significant alterations of liver function. Licensed preparations of entacapone contain entacapone alone or in combination with carbidopa and levodopa.

Levodopa preparations lead in the long term to the development of motor complications characterized by involuntary movements called dyskinesias and fluctuations in the response to medication. When this occurs a person with PD can change from phases with good response to medication and few symptoms (“on” state), to phases with no response to medication and significant motor symptoms (“off” state). For this reason, levodopa doses are kept as low as possible while maintaining functionality. Delaying the initiation of therapy with levodopa by using alternatives (dopamine agonists and MAO-B inhibitors) is common practice. A former strategy to reduce motor complications was to withdraw L-DOPA medication for some time. This is discouraged now since it can bring dangerous side effects such as neuroleptic malignant syndrome. Most people with PD will eventually need levodopa and later develop motor side effects.

Several dopamine agonists that bind to dopaminergic post-synaptic receptors in the brain have similar effects to levodopa. These were initially used for individuals experiencing on-off fluctuations and dyskinesias as a complementary therapy to levodopa; they are now mainly used on their own as an initial therapy for motor symptoms with the aim of delaying motor complications. When used in late PD they are useful at reducing the off periods. Dopamine agonists include bromocriptine, pergolide, pramipexole, ropinirole, piribedil, cabergoline, apomorphine and lisuride.

Dopamine agonists produce significant, although usually mild, side effects including drowsiness, hallucinations, insomnia, nausea, and constipation. Sometimes side effects appear even at a minimal clinically effective dose, leading the physician to search for a different drug. Compared with levodopa, dopamine agonists may delay motor complications of medication use, but are less effective at controlling symptoms. Nevertheless, they are usually effective enough to manage symptoms in the initial years. They tend to be more expensive than levodopa. Dyskinesias due to dopamine agonists are rare in younger people who have PD, but along with other side effects, become more common with age at onset. Thus, dopamine agonists are the preferred initial treatment for earlier onset, as opposed to levodopa in later onset. Agonists have been related to impulse control disorders (such as compulsive sexual activity and eating, and pathological gambling and shopping) even more strongly than levodopa.

Apomorphine, a non-orally administered dopamine agonist, may be used to reduce off periods and dyskinesia in late PD. It is administered by intermittent injections or continuous subcutaneous infusions. Since secondary effects such as confusion and hallucinations are common, individuals receiving apomorphine treatment should be closely monitored. Two dopamine agonists that are administered through skin patches (lisuride and rotigotine) and are useful for people in the initial stages and possibly to control off states in those in the advanced state.

MAO-B inhibitors (safinamide, selegiline and rasagiline) increase the level of dopamine in the basal ganglia by blocking its metabolism. They inhibit monoamine oxidase B (MAO-B) which breaks down dopamine secreted by the dopaminergic neurons. The reduction in MAO-B activity results in increased L-DOPA in the striatum. Like dopamine agonists, MAO-B inhibitors used as monotherapy improve motor symptoms and delay the need for levodopa in early disease but produce more adverse effects and are less effective than levodopa.

There are few studies of their effectiveness in the advanced stage, although results suggest that they are useful to reduce fluctuations between on and off periods. An initial study indicated that selegiline in combination with levodopa increased the risk of death, but this was later disproven.

Other drugs such as amantadine and anticholinergics may be useful as treatment of motor symptoms. However, the evidence supporting them lacks quality, so they are not first choice treatments. In addition to motor symptoms, PD is accompanied by a diverse range of symptoms. A number of drugs have been used to treat some of these problems. Examples are the use of quetiapine for psychosis, cholinesterase inhibitors for dementia, and modafinil for daytime sleepiness. A 2010 meta-analysis found that nonsteroidal anti-inflammatory drugs (apart from aspirin), have been associated with at least a 15 percent (higher in long-term and regular users) reduction of incidence of the development of Parkinson's disease

Treating motor symptoms with surgery was once a common practice, but since the discovery of levodopa, the number of operations declined. Studies in the past few decades have led to great improvements in surgical techniques, so that surgery is again being used in people with advanced PD for whom drug therapy is no longer sufficient. Surgery for PD can be divided in two main groups: lesional and deep brain stimulation (DBS). Target areas for DBS or lesions include the thalamus, the globus pallidus or the subthalamic nucleus. Deep brain stimulation is the most commonly used surgical treatment, developed in the 1980s by Alim Louis Benabid and others. It involves the implantation of a medical device called a neurostimulator, which sends electrical impulses to specific parts of the brain. DBS is recommended for people who have PD with motor fluctuations and tremor inadequately controlled by medication, or to those who are intolerant to medication, as long as they do not have severe neuropsychiatric problems. Other, less common, surgical therapies involve intentional formation of lesions to suppress overactivity of specific subcortical areas. For example, pallidotomy involves surgical destruction of the globus pallidus to control dyskinesia.

Exercise programs are recommended in people with Parkinson's disease. There is some evidence that speech or mobility problems can improve with rehabilitation, although studies are scarce and of low quality. Regular physical exercise with or without physical therapy can be beneficial to maintain and improve mobility, flexibility, strength, gait speed, and quality of life. When an exercise program is performed under the supervision of a physiotherapist, there are more improvements in motor symptoms, mental and emotional functions, daily living activities, and quality of life compared to a self-supervised exercise program at home. In terms of improving flexibility and range of motion for people experiencing rigidity, generalized relaxation techniques (e.g., gentle rocking) have been found to decrease excessive muscle tension. Other effective techniques to promote relaxation include slow rotational movements of the extremities and trunk, rhythmic initiation, diaphragmatic breathing, and meditation techniques. As for gait and addressing the challenges associated with the disease such as hypokinesia (slowness of movement), shuffling and decreased arm swing; physiotherapists have a variety of strategies to improve functional mobility and safety. Areas of interest with respect to gait during rehabilitation programs focus on, but are not limited to improving gait speed, the base of support, stride length, trunk and arm swing movement. Strategies include utilizing assistive equipment (pole walking and treadmill walking), verbal cueing (manual, visual and auditory), exercises (marching and PNF patterns) and altering environments (surfaces, inputs, open vs. closed). Strengthening exercises have shown improvements in strength and motor function for people with primary muscular weakness and weakness related to inactivity with mild to moderate Parkinson's disease. However, reports show a significant interaction between strength and the time the medications was taken. Therefore, it is recommended that people with PD should perform exercises 45 minutes to one hour after medications when they are at their best. Also, due to the forward flexed posture, and respiratory dysfunctions in advanced Parkinson's disease, deep diaphragmatic breathing exercises are beneficial in improving chest wall mobility and vital capacity. Exercise may improve constipation.

One of the most widely practiced treatments for speech disorders associated with Parkinson's disease is the Lee Silverman voice treatment (LSVT). Speech therapy and specifically LSVT may improve speech. Occupational therapy (OT) aims to promote health and quality of life by helping people with the disease to participate in as many of their daily living activities as possible. There have been few studies on the effectiveness of OT and their quality is poor, although there is some indication that it may improve motor skills and quality of life for the duration of the therapy.

Palliative care is specialized medical care for people with serious illnesses, including Parkinson's. The goal is to improve quality of life for both the person suffering from Parkinson's and the family by providing relief from the symptoms, pain, and stress of illnesses. As

Parkinson's is not a curable disease, all treatments are focused on slowing decline and improving quality of life and are therefore palliative in nature. Palliative care should be involved earlier, rather than later in the disease course. Palliative care specialists can help with physical symptoms, emotional factors such as loss of function and jobs, depression, fear, and existential concerns.

Along with offering emotional support to both the patient and family, palliative care serves an important role in addressing goals of care. People with Parkinson's may have many difficult decisions to make as the disease progresses such as wishes for feeding tube, non- invasive ventilator, and tracheostomy; wishes for or against cardiopulmonary resuscitation; and when to use hospice care. Palliative care team members can help answer questions and guide people with Parkinson's on these complex and emotional topics to help them make the best decision based on their own values.

Muscles and nerves that control the digestive process may be affected by PD, resulting in constipation and gastroparesis (food remaining in the stomach for a longer period than normal). A balanced diet, based on periodical nutritional assessments, is recommended and should be designed to avoid weight loss or gain and minimize consequences of gastrointestinal dysfunction. As the disease advances, swallowing difficulties (dysphagia) may appear. In such cases it may be helpful to use thickening agents for liquid intake and an upright posture when eating, both measures reducing the risk of choking. Gastrostomy to deliver food directly into the stomach is possible in severe cases.

Levodopa and proteins use the same transportation system in the intestine and the blood-brain barrier, thereby competing for access. When they are taken together, this results in a reduced effectiveness of the drug. Therefore, when levodopa is introduced, excessive protein consumption is discouraged and a well-balanced Mediterranean diet is recommended. In advanced stages, additional intake of low-protein products such as bread or pasta is recommended for similar reasons. To minimize interaction with proteins, levodopa should be taken 30 minutes before meals. At the same time, regimens for PD restrict proteins during breakfast and lunch, allowing protein intake in the evening.

Repetitive transcranial magnetic stimulation temporarily improves levodopa-induced dyskinesias. Its usefulness in PD is an open research topic, although recent studies have shown no effect by rTMS. Several nutrients have been proposed as possible treatments; however, there is no evidence that vitamins or food additives improve symptoms. There is no evidence to substantiate that acupuncture and practice of Qigong, or T′ai chi, have any effect on the course of the disease or symptoms. Further research on the viability of Tai chi for balance or motor skills are necessary. Fava beans and velvet beans are natural sources of levodopa and are eaten by many people with PD. While they have shown some effectiveness in clinical trials, their intake is not free of risks. Life-threatening adverse reactions have been described, such as the neuroleptic malignant syndrome.

PD invariably progresses with time. A severity rating method known as the Unified Parkinson's disease rating scale (UPDRS) is the most commonly used metric for clinical study. A modified version known as the MDS-UPDRS is also sometimes used. An older scaling method known as the Hoehn and Yahr scale (originally published in 1967), and a similar scale known as the Modified Hoehn and Yahr scale, have also been commonly used. The Hoehn & Yahr scale defines five basic stages of progression.

Motor symptoms, if not treated, advance aggressively in the early stages of the disease and more slowly later. Untreated, individuals are expected to lose independent ambulation after an average of eight years and be bedridden after ten years. However, it is uncommon to find untreated people nowadays. Medication has improved the prognosis of motor symptoms, while at the same time it is a new source of disability, because of the undesired effects of levodopa after years of use. In people taking levodopa, the progression time of symptoms to a stage of high dependency from caregivers may be over 15 years. However, it is hard to predict what course the disease will take for a given individual. Age is the best predictor of disease progression. The rate of motor decline is greater in those with less impairment at the time of diagnosis, while cognitive impairment is more frequent in those who are over 70 years of age at symptom onset.

Since current therapies improve motor symptoms, disability at present is mainly related to non-motor features of the disease. Nevertheless, the relationship between disease progression and disability is not linear. Disability is initially related to motor symptoms. As the disease advances, disability is more related to motor symptoms that do not respond adequately to medication, such as swallowing/speech difficulties and gait/balance problems. Motor complications also arise, which appear in up to 50% of individuals after 5 years of levodopa usage. Finally, after ten years most people with the disease have autonomic disturbances, sleep problems, mood alterations and cognitive decline. All of these symptoms, especially cognitive decline, greatly increase disability.

The life expectancy of people with PD is reduced. Mortality ratios are around twice those of unaffected people. Cognitive decline and dementia, old age at onset, a more advanced disease state and presence of swallowing problems are all mortality risk factors. On the other hand, a disease pattern mainly characterized by tremor as opposed to rigidity predicts an improved survival. Death from aspiration pneumonia is twice as common in individuals with PD as in the healthy population. In 2013 PD resulted in about 103,000 deaths globally, up from 44,000 deaths in 1990. The death rate increased from an average of 1.5 to 1.8 per 100,000 during that time.

### D. Amyotrophic Lateral Sclerosis

Amyotrophic lateral sclerosis (ALS), sometimes called Lou Gehrig's Disease, affects as many as 20,000 Americans at any given time, with 5,000 new cases being diagnosed in the United States each year. ALS affects people of all races and ethnic backgrounds. Men are about 1.5 times more likely than women to be diagnosed with the disease. ALS strikes in the prime of life, with people most commonly diagnosed between the ages of 40 and 70. However, it is possible for individuals to be diagnosed at younger and older ages. About 90-95% of ALS cases occur at random, meaning that individuals do not have a family history of the disease and other family members are not at increased risk for contracting the disease. In about 5-10% of ALS cases there is a family history of the disease.

ALS is a progressive neurological disease that attacks neurons that control voluntary muscles. Motor neurons, which are lost in ALS, are specialized nerve cells located in the brain, brainstem, and spinal cord. These neurons serve as connections from the nervous system to the muscles in the body, and their function is necessary for normal muscle movement. ALS causes motor neurons in both the brain and spinal cord to degenerate, and thus lose the ability to initiate and send messages to the muscles in the body. When the muscles become unable to function, they gradually atrophy and twitch. ALS can begin with very subtle symptoms such as weakness in affected muscles. Where this weakness first appears differs for different people, but the weakness and atrophy spread to other parts of the body as the disease progresses.

Initial symptoms may affect only one leg or arm, causing awkwardness and stumbling when walking or running Subjects also may suffer difficulty lifting objects or with tasks that require manual dexterity. Eventually, the individual will not be able to stand or walk or use hands and arms to perform activities of daily living. In later stages of the disease, when the muscles in the diaphragm and chest wall become too weak, patients require a ventilator to breathe. Most people with ALS die from respiratory failure, usually 3 to 5 years after being diagnosed; however, some people survive 10 or more years after diagnosis.

Perhaps the most tragic irony of ALS is that it does not impair a person's mind, as the disease affects only the motor neurons. Personality, intelligence, memory, and self-awareness are not affected, nor are the senses of sight, smell, touch, hearing, and taste. Yet at the same time, ALS causes dramatic defects in an individual's ability to speak loudly and clearly, and eventually, completely prevents speaking and vocalizing. Early speech-related symptoms include nasal speech quality, difficulty pronouncing words, and difficulty with conversation. As muscles for breathing weaken, it becomes difficult for patients to speak loud enough to be understood and, eventually, extensive muscle atrophy eliminates the ability to speak altogether. Patients also experience difficulty chewing and swallowing, which increase over time to the point that a feeding tube is required.

### E. Frontotemporal Dementia

Frontotemporal dementia (FTD), formerly known as disinhibition-dementia- parkinsonism-amyotrophy complex (DDPAC), is a neurodegenerative disease characterized by severe frontotemporal lobar degeneration. The disorder was first identified in 1994 by Kirk Wilhelmsen and colleagues, who distinguished it from Alzheimer's disease and Lewy body dementia based on the fact that it did not manifest with amyloid plaques, neurofibrillary tangles, or Lewy bodies. Second only to Alzheimer's disease (AD) in prevalence, FTD accounts for 20% of pre-senile dementia cases. Symptoms can begin to appear on average around 45 to 65 years of age, regardless of gender. The most common symptoms include significant changes in social and personal behavior, as well as a general blunting of emotions. Symptoms progress at a rapid, steady rate. Patients suffering from the disease can survive between 2-10 years.

Eventually patients will need 24-hour care for daily function. Because FTD often occurs in younger people (i.e., in their 40's or 50's), it can severely affect families Patients often still have children living in the home Financially, it can be devastating as the disease strikes at the time of life that is often the top wage-earning years. Currently, there is no cure for FTD. Treatments are available to manage the behavioral symptoms. Disinhibition and compulsive behaviors can be controlled by selective serotonin reuptake inhibitors (SSRIs). Although Alzheimer's and FTD share certain symptoms, they cannot be treated with the same pharmacological agents because the cholinergic systems are not affected in FTD.

FTD is traditionally difficult to diagnose due to the heterogeneity of the associated symptoms. Symptoms are classified into three groups based on the functions of the frontal and temporal lobes. Behavioral variant FTD (bvFTD) exhibits symptoms of lethargy and aspontaneity on the one hand, and disinhibition on the other. Apathetic patients may become socially withdrawn and stay in bed all day or no longer take care of themselves. Disinhibited patients can make inappropriate (sometimes sexual) comments or perform inappropriate acts. Patients with FTD can sometimes get into trouble with the law because of inappropriate behavior such as stealing or speeding. Recent findings indicate that psychotic symptoms are rare in FTD, possibly due to limited temporal-limbic involvement. Among FTD patients, approximately 2% have delusions, sometimes with paranoid ideation. Hallucinations are rare.

These psychotic symptoms are significantly less prevalent than what is seen in AD patients, where approximately 20% have delusions and paranoia. Progressive non-fluent aphasia (PNFA) presents with a breakdown in speech fluency due to articulation difficulty, phonological and/or syntactic errors but preservation of word comprehension. Semantic dementia (SD) can be found in some patients that remain fluent with normal phonology and syntax but increasing difficulty with naming and word comprehension. It has been researched that some may even go through depression and lose their inhibitions and exhibit antisocial behavior.

FTD patients tend to struggle with binge eating and compulsive behaviors. These binge eating habits are often associated with abnormal eating behavior including overeating, stuffing oneself with food, changes in food preferences (cravings for more sweets, carbohydrates), eating inedible objects and snatching food from others. Recent findings have indicated that the neural structures responsible for eating changes in FTD include atrophy in the right ventral insula, striatum and orbitofrontal cortex on structural MRI voxel-based morphometry (right hemisphere).

Executive function is the cognitive skill of planning and organizing. Most FTD patients become unable to perform skills that require complex planning or sequencing. In addition to the characteristic cognitive dysfunction, a number of primitive reflexes known as frontal release signs are often able to be elicited. Usually, the first of these frontal release signs to appear is the palmomental reflex which appears relatively early in the disease course whereas the palmar grasp reflex and rooting reflex appear late in the disease course. The following abilities in the FTD patients are preserved: perception, spatial skills, memory, and praxis. The following abilities in FTD patients are affected: social behavior/conduct, regulation of emotion, ability to focus, utilization behavior (neurobehavioral disorder where the patients grab objects in view and start to conduct the right behavior at the wrong time), and inappropriate speech/actions.

In rare cases, FTD can occur in patients with motor neuron disease (MND) (typically amyotrophic lateral sclerosis). The prognosis for people with MND is worse when combined with FTD, shortening survival by about a year. A number of case series have now been published looking at the pathological basis of frontotemporal dementia. As with other syndromes associated with frontotemporal lobar degeneration (FTLD), a number of different pathologies are associated with FTD:
Tau pathology: In a healthy individual, tau proteins stabilize microtubules, which are major component of the cytoskeleton. Examples include Picks disease, now also referred to as FTLD-tau, and other tau-positive pathology including FTDP-17, corticobasal degeneration, and progressive supranuclear palsy. Approximately 50% of FTD cases will present with tau pathology at post-mortem.
TDP-43 pathology: This disease form was previously described as dementia with ubiquitin positive, tau- and alpha-synuclein negative inclusions with and without motor neuron degeneration. FTLD-TDP43 accounts for approximately 40% of FTD (±MND).
FUS pathology: Cases with underlying FUS pathology tend to present with behavioural variant FTD (bvFTD), but the correlation is by no means reliable enough to predict the post mortem pathology. FTLD-FUS represents only 5-10% of clinically diagnosed FTD.
   Dementia lacking distinctive histology (DLDH) is a rare entity and represents the remaining small percentage of FTD that cannot be positively diagnosed as any of the above at post- mortem.
   In rare cases, patients with clinical FTD were found to have changes consistent with Alzheimer's disease on autopsy Evidence suggests that FTD selectively impairs spindle neurons, a type of neuron which has only been found in the brains of humans, great apes, and whales. Deficiencies of the micronutrients folate and B12 have been associated with cognitive impairment in individuals with FTD. Chronic folate deficiency has also been implicated in cerebral atrophy, leading to neurological impairment.

Structural MRI scans often reveal frontal lobe and/or anterior temporal lobe atrophy but in early cases the scan may seem normal. Atrophy can be either bilateral or asymmetric. Registration of images at different time points of time (e.g., one year apart) can show evidence of atrophy that otherwise (at individual time points) may be reported as normal. Many research groups have begun using techniques such as magnetic resonance spectroscopy, functional imaging and cortical thickness measurements in an attempt to offer an earlier diagnosis to the FTD patient. Fluorine-18-Fluorodeoxyglucose Positron Emission Tomography (FDG-PET) scans classically show frontal and/or anterior temporal hypometabolism, which helps differentiate the disease from Alzheimer's disease. The PET scan in Alzheimer's disease classically shows biparietal hypometabolism. Meta-analyses based on imaging methods have shown that frontotemporal dementia mainly affects a frontomedial network discussed in the context of social cognition or ‘theory of mind’. This is entirely in keeping with the notion that on the basis of cognitive neuropsychological evidence, the ventromedial prefrontal cortex is a major locus of dysfunction early on in the course of the behavioural variant of frontotemporal degeneration. The language subtypes of frontotemporal lobar degeneration (semantic dementia and progressive nonfluent aphasia) can be regionally dissociated by imaging approaches in vivo.

The confusion between Alzheimer's and FTD is justifiable due to the similarities between their initial symptoms. Patients do not have difficulty with movement and other motor tasks. As FTD symptoms appear, it is difficult to differentiate between a diagnosis of Alzheimer's disease and FTD. There are distinct differences in the behavioral and emotional symptoms of the two dementias, notably, the blunting of emotions seen in FTD patients. In the early stages of FTD, anxiety and depression are common, which may result in an ambiguous diagnosis. However, over time, these ambiguities fade away as this dementia progresses and defining symptoms of apathy, unique to FTD, start to appear.

In vivo brain imaging of tau aggregation in frontotemporal dementia using [¹⁸F]FDDNP positron emission tomography is more visual and has enhanced the ability to have a deeper understanding in frontal temporal dementia. Previous fluorescent microscopy studies of Alzheimer's disease (AD) brain specimens have shown that [¹⁸F]FDDNP displays an excellent visualization of interneuronal neurofibrillary tangles (NFTs). Visual images of [¹⁸F]FDDNP-PET images emphasized a frontal signal in FTD compared to prominent temporal signals in AD. [¹⁸F]FDDNP-PET has allowed the enhanced visualization of tauopathies in patients. This has aided in differentiating FTD from parietal and temporal signals in AD. Further, the ability of [¹⁸F]FDDNP to identify tauopathies in vivo gives a tool for monitoring the effect of therapies to eliminate NFT accumulation. Recent studies over several years have developed new criteria for the diagnosis of behavioral variant frontotemporal dementia (bvFTD). Six distinct clinical features have been identified as symptoms of bvFTD:
Disinhibition
Apathy/Inertia
Loss of Sympathy/Empathy
Perseverative/compulsive behaviors
Hyperorality
Dysexecutive neuropsychological profile
   Of the six features, three must be present in a patient to diagnose one with possible bvFTD. Similar to standard FTD, the primary diagnosis stems from clinical trials that identify the associated symptoms, instead of imaging studies. The above criteria are used to distinguish bvFTD from disorders such as Alzheimer's and other causes of dementia. In addition, the new criteria allow for a diagnostic hierarchy distinguished possible, probable, and definite bvFTD based on the number of symptoms present.

A higher proportion of FTD cases seem to have a familial component than more common neurodegenerative diseases like Alzheimer's disease. More and more mutations and genetic variants are being identified all the time, so the lists of genetic influences require consistent updating. Tau-positive frontotemporal dementia with parkinsonism (FTDP-17) is caused by mutations in the MAPT gene on chromosome 17 that encodes the Tau protein It has been determined that there is a direct relationship between the type of tau mutation and the neuropathology of gene mutations. The mutations at the splice junction of exon 10 of tau lead to the selective deposition of the repetative tau in neurons and glia. The pathological phenotype associated with mutations elsewhere in tau is less predictable with both typical neurofibrillary tangles (consisting of both 3 repeat and 4 repeat tau) and Pick bodies (consisting of 3 repeat tau) having been described). The presence of tau deposits within glia is also variable in families with mutations outside of exon 10. This disease is now informally designated FTDP-17T. FTD shows a linkage to the region of the tau locus on chromosome 17, but it is believed that there are two loci leading to FTD within megabases of each other on chromosome 17. FTD caused by FTLD-TDP43 has numerous genetic causes. Some cases are due to mutations in the GRN gene, also located on chromosome 17. Others are caused by VCP mutations, although these patients present with a complex mixture of Inclusion body myopathy, Paget's disease of bone, and FTD. The most recent addition to the list is a hexanucleotide repeat expansion in the promotor region of C9ORF72. Only one or two cases have been reported describing TARDBP (the TDP-43 gene) mutations in a clinically pure FTD (FTD without MND).

### III. Methods for Inhibiting Protein Aggregation

In accordance with the present disclosure, the inventors contemplate the use of SRRD or active fragment thereof in methods to inhibit protein aggregation both in vitro and in vivo. In particular, in vivo embodiments are disclosed for use in the treatment of neurologic disease characterized by protein aggregates, such as AD, PD, FTD and ALS. SRRD may be delivered itself to the cell or subject or may be produced in the cell or subject following delivery of an expression construct the encodes the SRRD gene or active fragment thereof and contains sufficient elements to support expression of SRRD or active fragment thereof in vitro or in vivo. The following passages describe relevant supporting technologies.

### A. Protein Delivery

In one embodiment, SRRD or active fragments thereof may be delivered to a cell or subject. The SRRD or active fragment thereof may be formulated in a carrier without any other agents, or it may be further complexed with delivery materials such as lipids (e.g., in a liposome) or nanoparticles (lipid, noble metal, etc.). It may also prove useful to conjugate SRRD or active fragment thereof to another molecule, such as a molecule that stabilizes SRRD or active fragment thereof, prevents degradation of SRRD or active fragment thereof, permits monitoring of SRRD or active fragment thereof delivery to the CNS, facilities traversal of the blood-brain barrier by SRRD or active fragment thereof and/or facilities uptake of SRRD or active fragment thereof by target cells. Some of these technologies are described below.

Cell-penetrating peptides (CPPs) are short peptides that facilitate cellular intake/uptake of various molecular equipment (from nanosize particles to small chemical molecules and large fragments of DNA). The “cargo” is associated with the peptides either through chemical linkage via covalent bonds or through non-covalent interactions. The function of the CPPs are to deliver the cargo into cells, a process that commonly occurs through endocytosis with the cargo delivered to delivery vectors for use in research and medicine. Current use is limited by a lack of cell specificity in CPP-mediated cargo delivery and insufficient understanding of the modes of their uptake, that is why other delivery mechanisms have been developed like CellSqueeze and electroporation.

CPPs typically have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake.

### 1. CPPs

The first CPP was discovered independently by two laboratories in 1988, when it was found that the trans-activating transcriptional activator (TAT) from human immunodeficiency virus 1 (HIV-1) could be efficiently taken up from the surrounding media by numerous cell types in culture. Since then, the number of known CPPs has expanded considerably and small molecule synthetic analogues with more effective protein transduction properties have been generated. A recent discovery found that Papillomaviridae such as the Human Papillomavirus use CPPs to penetrate the intracellular membrane in order to trigger retrograde trafficking of the viral unit to the nucleus.

Cell-penetrating peptides are of different sizes, amino acid sequences, and charges but all CPPs have one distinct characteristic, which is the ability to translocate the plasma membrane and facilitate the delivery of various molecular cargoes to the cytoplasm or an organelle. There has been no real consensus as to the mechanism of CPP translocation, but the theories of CPP translocation can be classified into three main entry mechanisms: direct penetration in the membrane, endocytosis-mediated entry, and translocation through the formation of a transitory structure. CPP transduction is an area of ongoing research. Cell-penetrating peptides (CPP) are able to transport different types of cargo molecules across plasma membrane; thus, they act as molecular delivery vehicles. They have numerous applications in medicine as drug delivery agents in the treatment of different diseases including cancer and virus inhibitors, as well as contrast agents for cell labeling. Examples of the latter include acting as a carrier for GFP, MRI contrast agents, or quantum dots.

### 2. Liposomes

Liposomes are artificially prepared vesicles made of lipid bilayers have been used to deliver a variety of drugs. Liposomes can be composed of naturally derived phospholipids with mixed lipid chains (like egg phosphatidylethanolamine) or other surfactants. In particular, liposomes containing cationic or neutural lipids have been used in the formulation of drugs.

Liposomes should not be confused with micelles and reverse micelles composed of monolayers, which also can be used for delivery. A wide variety of commercial formulations for protein delivery are well known including PULSin®, Lipodin-Pro, Carry-MaxR, Pro-DeliverIN, PromoFectin, Pro-Ject, Chariot® Protein Delivery reagent, BioPORTER®, and others.

### 3. Nanoparticles

In nanotechnology, a particle is defined as a small object that behaves as a whole unit with respect to its transport and properties. Particles are further classified according to diameter, with those under 1 μm in size being nanoparticles, but more generally between 1 and 100 nanometers in size. Nanoparticles can be made of a variety of different materials, such as proteins, lipids, polymers and metals. Depending on their composition, synthesis and use, they may carry deliverable molecules on their surface or internally.

In contrast to liposomes, which are hollow, nanoparticles tend to be solid. Thus, the agent will be less entrapped and more either embedded in or coated on the nanoparticle. Nanoparticles can be made of metals including oxides, silica, polymers such as polymethyl methacrylate, lipids and ceramics. Similarly, nanoshells are somewhat larger and encase the delivered substances with these same materials. Either nanoparticles or nanoshells permit sustained or controlled release of the agent and can stabilize it to the effects of in vivo environment.

B. Genetic Delivery In another embodiment, the inventors envision employing expression constructs encoding SRRD or active fragment thereof for delivery and expression by a transformed cell. There are a number of ways in which such expression vectors may introduced into cells. Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran

(Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). Some of these are discussed in greater detail below.

### 1. AAV

Adeno associated virus (AAV) is a small nonpathogenic virus of the parvoviridae family To date, numerous serologically distinct AAVs have been identified, and more than a dozen have been isolated from humans or primates. AAV is distinct from the other members of this family by its dependence upon a helper virus for replication.

AAV genomes been shown to stably integrate into host cellular genomes; possess a broad host range; transduce both dividing and non-dividing cells in vitro and in vivo and maintain high levels of expression of the transduced genes. AAV viral particles are heat stable, resistant to solvents, detergents, changes in pH, temperature, and can be concentrated on CsCl gradients or by other means. The AAV genome comprises a single- stranded deoxyribonucleic acid (ssDNA), either positive- or negative-sensed. In the absence of a helper virus, AAV may integrate in a locus specific manner, for example into the q arm of chromosome 19. The approximately 5 kb genome of AAV consists of one segment of single stranded DNA of either plus or minus polarity. The ends of the genome are short inverted terminal repeats which can fold into hairpin structures and serve as the origin of viral DNA replication.

An AAV “genome” refers to a recombinant nucleic acid sequence that is ultimately packaged or encapsulated to form an AAV particle. An AAV particle often comprises an AAV genome. In cases where recombinant plasmids are used to construct or manufacture recombinant vectors, the vector genome does not include the portion of the “plasmid” that does not correspond to the vector genome sequence of the recombinant plasmid. This non-vector genome portion of the recombinant plasmid is referred to as the “plasmid backbone,” which is important for cloning and amplification of the plasmid, a process that is needed for propagation and recombinant virus production but is not itself packaged or encapsulated into virus (e.g.,

AAV) particles. Thus, a vector “genome” refers to nucleic acid that is packaged or encapsulated by virus (e.g., AAV).

A viral vector is derived from or based upon one or more nucleic acid elements that comprise a viral genome. Particular viral vectors include adeno-associated virus (AAV) vectors. Also provided are vectors (e.g., AAV) comprising a nucleic acid sequence encoding SRRD, variant or subsequence (e.g., a polypeptide fragment having SRRD activity). The term “recombinant” as a modifier of vector, such as recombinant viral, e.g., lenti- or parvo-virus (e.g., AAV) vectors, as well as a modifier of sequences such as recombinant polynucleotides and polypeptides, means that the compositions have been manipulated (i.e., engineered) in a fashion that generally does not occur in nature. A particular example of a recombinant vector, such as an AAV vector would be where a polynucleotide that is not normally present in the wild-type viral (e.g., AAV) genome is inserted within the viral genome. An example of a recombinant polynucleotide would be where a nucleic acid (e.g., gene) encoding SRRD is cloned into a vector, with or without 5′, 3′ and/or intron regions that the gene is normally associated within the viral (e.g., AAV) genome. Although the term “recombinant” is not always used herein in reference to vectors, such as viral and AAV vectors, as well as sequences such as polynucleotides, recombinant forms including polynucleotides, are expressly included in spite of any such omission.

The AAV virion (particle) is a non-enveloped, icosahedral particle approximately 25 nm in diameter. The AAV particle comprises a capsid of icosahedral symmetry comprised of three related capsid proteins, VP1, VP2 and VP3, which interact together to form the capsid. The right ORF often encodes the capsid proteins VP1, VP2, and VP3. These proteins are often found in a ratio of 1:1:10 respectively, but may be in varied ratios, and are all derived from the right-hand ORF. The capsid proteins differ from each other by the use of alternative splicing and an unusual start codon. Deletion analysis has shown that removal or alteration of VP1 which is translated from an alternatively spliced message results in a reduced yield of infectious particles. Mutations within the VP3 coding region result in the failure to produce any single- stranded progeny DNA or infectious particles. An AAV particle is a viral particle comprising an AAV capsid. In certain embodiments the genome of an AAV particle encodes one, two or all VP1, VP2 and VP3 polypeptides.

The genome of most native AAVs often contain two open reading frames (ORFs), sometimes referred to as a left ORF and a right ORF. The left ORF often encodes the nonstructural Rep proteins, Rep 40, Rep 52, Rep 68 and Rep 78, which are involved in regulation of replication and transcription in addition to the production of single-stranded progeny genomes. Two of the Rep proteins have been associated with the preferential integration of AAV genomes into a region of the q arm of human chromosome 19. Rep68/78 have been shown to possess NTP binding activity as well as DNA and RNA helicase activities. Some Rep proteins possess a nuclear localization signal as well as several potential phosphorylation sites. In certain embodiments the genome of an AAV (e.g., an rAAV) encodes some or all of the Rep proteins. In certain embodiments the genome of an AAV (e.g., an rAAV) does not encode the Rep proteins. In certain embodiments one or more of the Rep proteins can be delivered in trans and are therefore not included in an AAV particle comprising a nucleic acid encoding a polypeptide. The ends of the AAV genome comprise short inverted terminal repeats (ITR) which have the potential to fold into T-shaped hairpin structures that serve as the origin of viral DNA replication. Accordingly, the genome of an AAV comprises one or more (e.g., a pair of) ITR sequences that flank its single stranded viral DNA genome. The ITR sequences often comprise about 145 bases each. Within the ITR region, two elements have been described which are thought to be central to the function of the ITR, a GAGC repeat motif and the terminal resolution site (trs). The repeat motif has been shown to bind Rep when the ITR is in either a linear or hairpin conformation. This binding is thought to position Rep68/78 for cleavage at the trs which occurs in a site- and strand- specific manner In addition to their role in replication, these two elements appear to be central to viral integration. Contained within the chromosome 19 integration locus is a Rep binding site with an adjacent trs. These elements have been shown to be functional and necessary for locus specific integration.

In certain embodiments an AAV (e.g., an rAAV) comprises two ITRs. In certain embodiments an AAV (e.g., an rAAV) comprises a pair of ITRs. In certain embodiments an AAV (e.g., an rAAV) comprises a pair of ITRs that flank (i.e., are at each 5′ and 3′ end) of a polynucleotide that at least encodes SRRD.

A recombinant viral “vector” or “AAV vector” is derived from the wild type genome of a virus, such as AAV by using molecular methods to remove the wild type genome from the virus (e.g., AAV), and replacing with a non-native nucleic acid, such as a SRRD-encoding nucleic acid sequence. Typically, for AAV one or both inverted terminal repeat (ITR) sequences of AAV genome are retained in the AAV vector. A “recombinant” viral vector (e.g., rAAV) is distinguished from a viral (e.g., AAV) genome, since all or a part of the viral genome has been replaced with a non-native sequence with respect to the viral (e.g., AAV) genomic nucleic acid such as SRRD-encoding nucleic acid sequence. Incorporation of a non-native sequence therefore defines the viral vector (e.g., AAV) as a “recombinant” vector, which in the case of AAV can be referred to as a “rAAV vector.”

An AAV vector (e.g., rAAV vector) can be packaged and is referred to herein as an “AAV particle” for subsequent infection (transduction) of a cell, ex vivo, in vitro or in vivo. Where a recombinant AAV vector is encapsulated or packaged into an AAV particle, the particle can also be referred to as a “rAAV particle.” In certain embodiments, an AAV particle is an rAAV particle. A rAAV particle often comprises an AAV vector, or a portion thereof. A rAAV particle can be one or more AAV particles (e.g., a plurality of AAV particles). rAAV particles typically comprise proteins that encapsulate or package the rAAV vector genome (e.g., capsid proteins). Any suitable AAV particle (e.g., rAAV particle) can be used for a method or use herein. A rAAV particle, and/or genome comprised therein, can be derived from any suitable serotype or strain of AAV. A rAAV particle, and/or genome comprised therein, can be derived from two or more serotypes or strains of AAV. Accordingly, a rAAV can comprise proteins and/or nucleic acids, or portions thereof, of any serotype or strain of AAV, wherein the AAV particle is suitable for infection and/or transduction of a mammalian cell. Non-limiting examples of AAV serotypes include AAV1, AAV2, AAV3, AAV4, AAV5,

AAV6, AAV7, AAV8, AAV9, AAV10, AAV11, AAV 12, AAV-rh74, AAV-rh1O or AAV-2i8. In certain embodiments a plurality of rAAV particles comprises particles of, or derived from, the same strain or serotype (or subgroup or variant). In certain embodiments a plurality of rAAV particles comprise a mixture of two or more different rAAV particles (e.g., of different serotypes and/or strains).

As used herein, the term “serotype” is a distinction used to refer to an AAV having a capsid that is serologically distinct from other AAV serotypes. Serologic distinctiveness is determined on the basis of the lack of cross -reactivity between antibodies to one AAV as compared to another AAV. Such cross -reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

In certain embodiments, a rAAV vector based upon a first serotype genome is identical to the serotype of one or more of the capsid proteins that package the vector. In certain embodiments, a rAAV vector genome can be based upon an AAV (e.g., AAV2) serotype genome distinct from the serotype of one or more of the AAV capsid proteins that package the vector. For example, a rAAV vector genome can comprise AAV2 derived nucleic acids (e.g., ITRs), whereas at least one or more of the three capsid proteins are derived from a different serotype, e.g., a AAV1, AAV3, AAV4, AAV5, AAV6, AAV7, AAV8, AAV9, AAV10, AAV11, AAV 12, RhIO, Rh74 or AAV-2i8 serotype or variant thereof.

Recombinant AAV vectors that include a polynucleotide that directs the expression of a polypeptide can be generated using suitable recombinant techniques known in the art (e.g., see Sambrook et al., 1989). Recombinant AAV vectors are typically packaged into transduction- competent AAV particles and propagated using an AAV viral packaging system. A transduction-competent AAV particle is capable of binding to and entering a mammalian cell and subsequently delivering a nucleic acid cargo (e.g., a heterologous gene) to the nucleus of the cell. Thus, an intact AAV particle that is transduction-competent is configured to transduce a mammalian cell. An AAV particle configured to transduce a mammalian cell is often not replication competent and requires additional protein machinery to self-replicate. Thus, an AAV particle that is configured to transduce a mammalian cell is engineered to bind and enter a mammalian cell and deliver a nucleic acid to the cell, wherein the nucleic acid for delivery is often positioned between a pair of AAV ITRs in the AAV genome.

Suitable host cells for producing transduction-competent AAV particles include but are not limited to microorganisms, yeast cells, insect cells, and mammalian cells that can be, or have been, used as recipients of a heterologous rAAV vectors. Cells from the stable human cell line, 293 (readily available through, e.g., the American Type Culture Collection under Accession Number ATCC CRL1573) can be used. In certain embodiments a modified human embryonic kidney cell line (e.g., HEK293), which is transformed with adenovirus type-5 DNA fragments and expresses the adenoviral Ela and Elb genes is used to generate recombinant AAV particles. The modified HEK293 cell line is readily transfected and provides a particularly convenient platform in which to produce rAAV particles. Methods of generating high titer AAV particles capable of transducing mammalian cells are known in the art. For example, AAV particle can be made as set forth in Wright, 2008 and Wright, 2009.

In certain embodiments, AAV helper functions are introduced into the host cell by transfecting the host cell with an AAV helper construct either prior to, or concurrently with, the transfection of an AAV expression vector. AAV helper constructs are thus sometimes used to provide at least transient expression of AAV rep and/or cap genes to complement missing

AAV functions necessary for productive AAV transduction. AAV helper constructs often lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products.

In certain embodiments, an AAV particle or a vector genome thereof related to a reference serotype has a polynucleotide, polypeptide or subsequence thereof that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a polynucleotide, polypeptide or subsequence of an AAV strain. In particular embodiments, an AAV particle or a vector genome thereof related to a reference serotype has a capsid or ITR sequence that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a capsid or ITR sequence of an AAV strain.

In certain embodiments, a rAAV particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV strain, as long as they retain one or more desired ITR functions (e.g., ability to form a hairpin, which allows DNA replication; integration of the AAV DNA into a host cell genome; and/or packaging, if desired).

A rAAV particle can comprise an ITR having any suitable number of “GAGC” repeats. In certain embodiments an ITR of an AAV particle comprises 1, 2, 3, 4, 5, 6, 7, 8, 9 or 10 or more “GAGC” repeats. In certain embodiments a rAAV particle comprises an ITR comprising three “GAGC” repeats. In certain embodiments a rAAV particle comprises an ITR which has less than four “GAGC” repeats. In certain embodiments a rAAV particle comprises an ITR which has more than four “GAGC” repeats. In certain embodiments an ITR of a rAAV particle comprises a Rep binding site wherein the fourth nucleotide in the first two “GAGC” repeats is a C rather than a T.

Any suitable length of DNA can be incorporated into an AAV particle. Suitable DNA molecules for use in rAAV vectors can about 5 kilobases (kb), less than about 5kb, less than about 4.5 kb, less than about 4 kb, less than about 3.5 kb, less than about 3 kb, or less than about 2.5 kb.

A “transgene” is used herein to conveniently refer to a nucleic acid that is intended or has been introduced into a cell or organism. Transgenes include any nucleic acid, such as a gene that encodes a polypeptide or protein (e.g., SRRD), and are generally heterologous with respect to naturally occurring AAV genomic sequences.

In a cell having a transgene, the transgene is often introduced/transferred by way of a vector, such as a rAAV particle. Introduction of a transgene into a cell by a rAAV particle is often referred to as “transduction” of the cell. The term “transduce” refers to introduction of a molecule such as a nucleic acid into a cell or host organism by way of a vector (e.g., an AAV particle). The transgene may or may not be integrated into genomic nucleic acid of a transduced cell. If an introduced nucleic acid becomes integrated into the nucleic acid (genomic DNA) of the recipient cell or organism it can be stably maintained in that cell or organism and further passed on to or inherited by progeny cells or organisms of the recipient cell or organism. Finally, the introduced nucleic acid may exist in the recipient cell or host organism extra chromosomally, or only transiently. A “transduced cell” is a cell into which the transgene has been introduced by way of transduction. Thus, a “transduced” cell is a cell into which, or a progeny thereof in which a nucleic acid has been introduced. A transduced cell can be propagated and the introduced protein expressed, or nucleic acid transcribed. For gene therapy uses and methods, a transduced cell can be in a mammal.

Nucleic acids can include one or more expression control or regulatory elements operably linked to the open reading frame, where the one or more regulatory elements are configured to direct the transcription and translation of the polypeptide encoded by the open reading frame in a mammalian cell. Non-limiting examples of expression control/regulatory elements include transcription initiation sequences (e.g., promoters, enhancers, a TATA box, and the like), translation initiation sequences, mRNA stability sequences, poly A sequences, secretory sequences, and the like. Expression control/regulatory elements can be obtained from the genome of any suitable organism. Non-limiting examples include SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from non-viral genes, such as the murine metallothionein gene, will also find use herein.

Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes under control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP.

Promoters containing a particular RE can be chosen in order to obtain an inducible response and in some cases, the RE itself may be attached to a different promoter, thereby conferring inducibility to the recombinant gene. Thus, by selecting a suitable promoter (constitutive versus inducible; strong versus weak), it is possible to control both the existence and level of expression of a polypeptide in the genetically modified cell. If the gene encoding the polypeptide is under the control of an inducible promoter, delivery of the polypeptide in situ is triggered by exposing the genetically modified cell in situ to conditions for permitting transcription of the polypeptide, e.g., by intraperitoneal injection of specific inducers of the inducible promoters which control transcription of the agent. For example, in situ expression by genetically modified cells of a polypeptide encoded by a gene under the control of the metallothionein promoter, is enhanced by contacting the genetically modified cells with a solution containing the appropriate (i.e., inducing) metal ions in situ.

A nucleic acid is “operably linked” when it is placed into a functional relationship with another nucleic acid sequence. A nucleic acid encoding a polypeptide, or a nucleic acid directing expression of a SRRD polypeptide (e.g., a polypeptide having SRRD activity) may include an inducible promoter, or a tissue- specific promoter for controlling transcription of the encoded polypeptide.

In certain embodiments, an expression control element comprises a CMV enhancer. In certain embodiments, an expression control element comprises a beta actin promoter. In certain embodiments, an expression control element comprises a chicken beta actin promoter. In certain embodiments, an expression control element comprises a CMV enhancer and a chicken beta actin promoter.

### 2. Adenovirus

“Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid- sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The El region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

### 3. Retroviruses

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

### 4. Other Viral Vectors

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

### C. Pharmaceutical Formulations

Clinical applications according the present disclosure require preparing of pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render materials stable and allow for uptake by target cells. Aqueous compositions of the present disclosure comprise an effective amount of the protein or vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the protein or vectors of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intramuscular, intraperitoneal, intracranial, intrathecal or intravenous injection. Such compositions would normally be administered as pharmaceutically acceptable compositions, described supra. The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active agent in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present disclosure may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### D. Combination Therapies

In certain embodiments, the compositions and methods of the present embodiments involve the use of SRRD or active fragment thereof or expression vector coding therefor in combination with at least one additional therapy. The additional therapy may be any of those referenced above in the section on disease states.

The SRRD or active fragment thereof or expression vector coding therefor may be administered before, during, after, or in various combinations relative to an additional cancer therapy, such as immune checkpoint therapy. The administrations may be in intervals ranging from concurrently to minutes to days to weeks. In some embodiments where the SRRD or active fragment thereof or expression vector coding therefor is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below the SRRD or active fragment thereof or expression vector coding therefor is “A” and the other therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

CRISPR screen. 293T cells were transduced with lentiviral vectors containing the Brunello CRISPR library (one vector system containing both Cas9 and sgRNA) (world-wide- web at addgene.org/pooled-library/broadgpp-human-knockout-brunello/) at a 1000x coverage (cells/sgRNA) and at a low (0.4) Multiplicity Of Infection (MOI). Cells are selected with puromycin and cultured for 10 days to maximize gene knockout efficiency. At this point cells are transiently transfected with an inducible TDP-43 aggregating construct. Cells are fixed after two days and sorted using a BD Aria Fusion instrument over a period of 2-3 weeks to have enough sorted cells that will maintain 1000x coverage over library complexity. Sorted cells are proceeded for gDNA extraction and NGS library preparation as described below.

gDNA extraction. For 3×10⁷−5×10⁷ of frozen cell pellet the same procedure is used. For different amounts of tissue or cells, the quantities were scaled proportionally. In a 15 ml conical tube, 6 ml of NK Lysis Buffer (50 mM Tris, 50 mM EDTA, 1% SDS, pH 8) and 30 pl of 20 mg/ml Proteinase K (Qiagen 19131) were added to the tissue/cell sample and incubated at 55° C. overnight. The next day, 30 μl of 10 mg/ml RNAse A (Qiagen 19101, diluted in NK Lysis Buffer to 10 mg/ml and then stored at 4° C.) was added to the lysed sample, which was then inverted 25 times and incubated at 37° C. for 30 minutes. Samples were cooled on ice before addition of 2 ml of pre-chilled 7.5 M ammonium acetate (Sigma A1542) to precipitate proteins. Stock solutions of 7.5 M ammonium acetate was made in sterile dH2O and kept at 4° C. until use. After adding ammonium acetate, the samples were vortexed at high speed for 20 seconds and then centrifuged at >4,000×g for 10 minutes. After the spin, a tight pellet was visible in each tube and the supernatant was carefully decanted into a new 15 ml conical tube. Then 6 ml 100% isopropanol was added to the tube, inverted 50 times and centrifuged at >4,000 ×g for 10 minutes. Genomic DNA was visible as a small white pellet in each tube. The supernatant was discarded, 6 ml of freshly prepared 70% ethanol was added, the tube was inverted 10 times, and then centrifuged at >4,000×g for 1 minute. The supernatant was discarded by pouring; the tube was briefly spun, and remaining ethanol was removed using a P200 pipette. After air drying for 10-30 minutes, the DNA changed appearance from a milky white pellet to slightly translucent. At this stage, 500 μl of 1× TE buffer (Sigma T9285) was added, the tube was incubated at 65° C. for 1 hour and at room temperature overnight to fully resuspend the DNA. The next day, the gDNA samples were vortexed briefly. The gDNA concentration was measured using a Nanodrop (Thermo Scientific).

Sequencing Library Preparation. The inventors performed targeted PCR to amplify the integrated lentiviral cassette and use next generation sequencing to quantify sgRNA frequencies between the non-aggregating and aggregating samples. PCR was performed on all the gDNA extracted divided into 100 μl reactions with 5 μg of gDNA in each using the TaKara ExTaq enzyme. PCR mix was assembled using manufacturer instructions. The used the following barcoded primers:

Fw primer mix:
(SEQ ID NO: 2)
AATGATACGGCGACCACCGAGATCTACACTCTTTCCCTACACGACGCTCT
TCCGATCTtTTGTGGAAAGGACGAAACACCG
(SEQ ID NO: 3)
AATGATACGGCGACCACCGAGATCTACACTCTTTCCCTACACGACGCTCT
TCCGATCTatTTGTGGAAAGGACGAAACACCG
(SEQ ID NO: 4)
AATGATACGGCGACCACCGAGATCTACACTCTTTCCCTACACGACGCTCT
TCCGATCTgatTTGTGGAAAGGACGAAACACCG
(SEQ ID NO: 5)
AATGATACGGCGACCACCGAGATCTACACTCTTTCCCTACACGACGCTCT
TCCGATCTcgatTTGTGGAAAGGACGAAACACCG
(SEQ ID NO: 6)
AATGATACGGCGACCACCGAGATCTACACTCTTTCCCTACACGACGCTCT
TCCGATCTtcgatTTGTGGAAAGGACGAAACACCG
(SEQ ID NO: 7)
AATGATACGGCGACCACCGAGATCTACACTCTTTCCCTACACGACGCTCT
TCCGATCTatcgatTTGTGGAAAGGACGAAACACCG
(SEQ ID NO: 8)
AATGATACGGCGACCACCGAGATCTACACTCTTTCCCTACACGACGCTCT
TCCGATCTgatcgatTTGTGGAAAGGACGAAACACCG
(SEQ ID NO: 9)
AATGATACGGCGACCACCGAGATCTACACTCTTTCCCTACACGACGCTCT
TCCGATCTcgatcgatTTGTGGAAAGGACGAAACACCG
(stagger used to increase complexity)
Rv primer:
<8bp sample barcode>
(SEQ ID NO: 10)
CAAGCAGAAGACGGCATACGAGATA
(SEQ ID NO: 11)
TGACTGGAGTTCAGACGTGTGCTCTTCCGATCTCCGACTCGGTGCCACTT
TTTCAA

Data Analysis. Sequencing data was analyzed using our customized computational pipeline written in R programming language. Briefly, the inventors used a regular expression to extract the sgRNA sequence out of each sequencing read. They then used Bowtie to map those sequences to a prebuild library index. Read counts were then normalized reads per million +1 and the ratio between samples for each sgRNA was calculated. In order to correct mean to variance relationship, the inventors calculated a local Z score for each sgRNA, which is the Z score of the 2000 sgRNAs with the closest mean (across the two samples) using a sliding window. Gene based p-values were calculated by comparing the mean sgRNA local Z score compared to an empirical distribution calculated using 100,000 data permutations. Using this approach, the inventors identified SRRD as one of our top gene hits in the screen.

### Example 2—Results

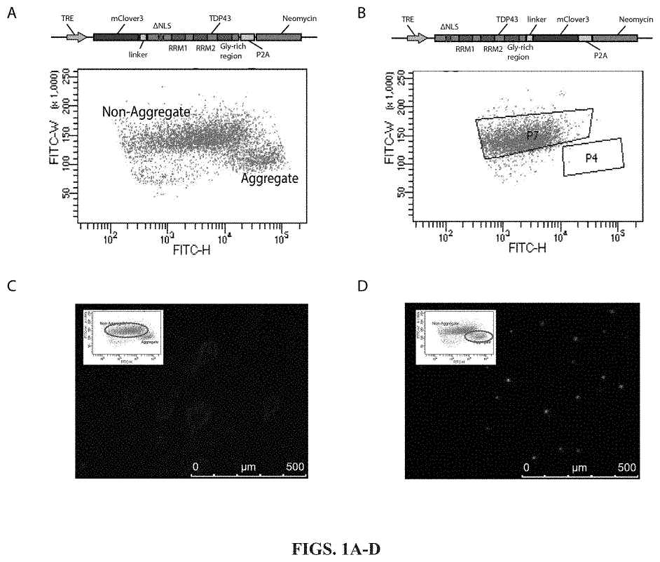

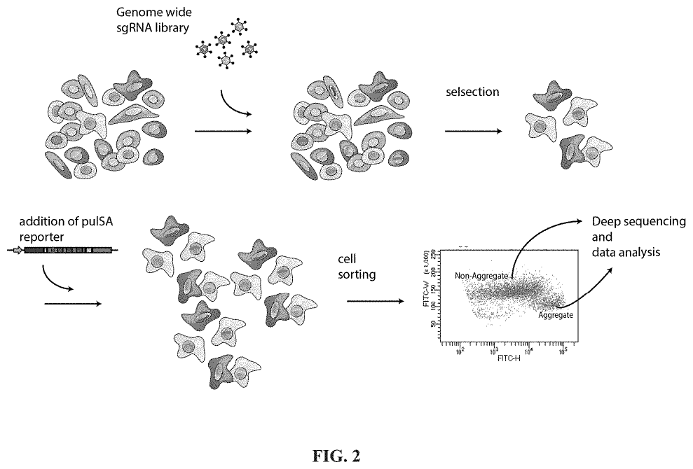

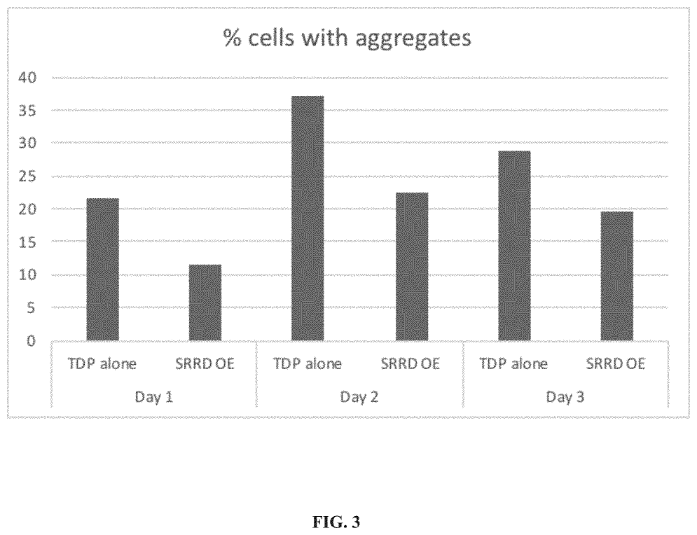

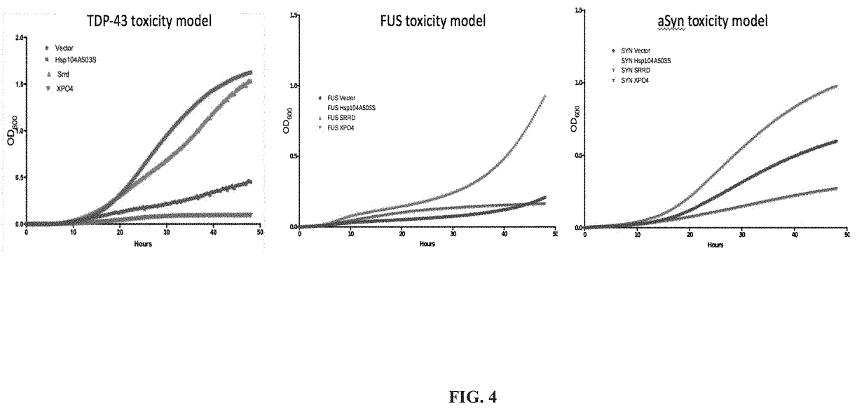

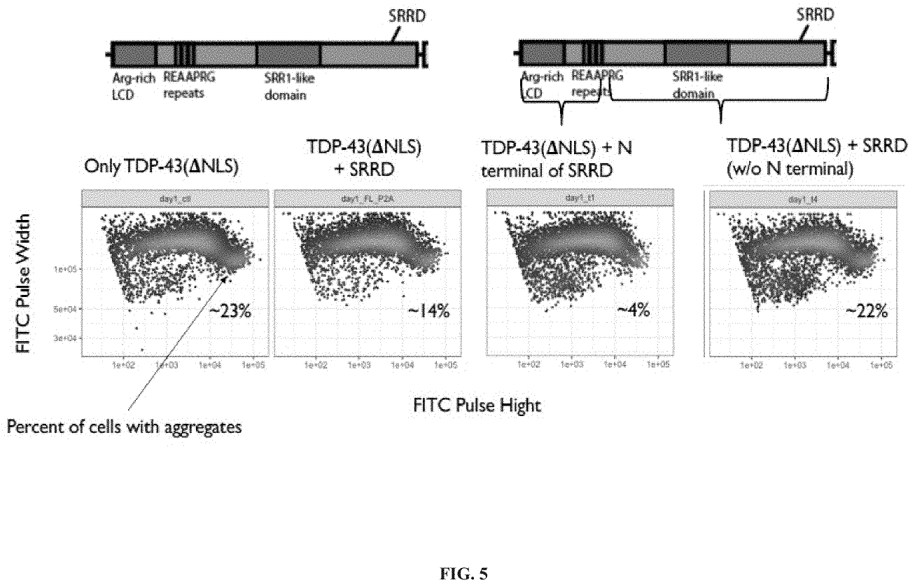

The inventors used an overexpression model of a mutant TDP-43 with deleted NLS that forms visible aggregates in mammalian cells coupled with a FACS-based readout that their lab has developed (FIG. 1). They then performed a genome-wide CRISPR screens to identify proteins that when in-activated result in either increase or decrease of the % of cells with aggregates (FIG. 2). As one of the strongest gene hits they identified a protein called SRRD. They found that when overexpressing SRRD there is reduction of protein aggregation in a TDP-43 human cell line model (FIG. 3) and very strong reduced toxicity in a yeast TDP-43 toxicity model (FIG. 4). Interestingly, overexpression of SRRD was also protective against other unfolded proteins in yeast including Alpha Synuclein which is associated with PD and FUS (FIG. 4). FIG. 5 shows that SRRD is a novel modifier that affects aggregation through its N-terminal Arg-rich LCD.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. A method of reducing protein aggregation in a cell comprising providing to a cell SRR1 domain-containing protein (SRRD) or active fragment thereof.

2. The method of claim 1, wherein providing comprises contacting said cell with SRRD or active fragment thereof.

3. The method of claim 1, wherein providing comprises contacting said cell with an expression construct encoding SRRD or active fragment thereof.

4. The method of claim 3, wherein said expression construct is a viral expression construct, such as an adeno-associated virus construct.

5. The method of claim 3, wherein said expression construct is a non-viral expression construct.

6. The method of claims 1-5, wherein said SRRD or active fragment thereof further comprises a cell permeability peptide.

7. The method of claims 2-6, wherein said SRRD or active fragment thereof or expression construct encoding SSRD or active fragment thereof is contacted with said cell in a liposome or nanoparticle.

8. The method of claims 1-7, wherein said cell is a eukaryotic cell.

9. The method of claims 1-7, wherein said cell is a mammalian cell, such as a human cell.

10. The method of claims 1-7, wherein said cell is a yeast cell.

11. The method of claims 1-10, wherein SRRD or active fragment thereof is provided to said cell more than once.

12. The method of claims 1-11, further comprising assessing protein aggregation in said cell prior to and/or after said providing.

13. The method of claims 1-12, further comprising providing to said cell a second anti- protein aggregation treatment.

14. The method of claims 1-13, wherein SRRD or active fragment thereof is provided at a level higher than normally expressed in said cell.

15. The method of claims 1-14, wherein said cell is a mammalian cell and is located in a living mammal.

16. A method of treating a subject suffering from a protein aggregation disease or disorder comprising providing to said subject SRR1 domain-containing protein (SRRD) or active fragment thereof.

17. The method of claim 16, wherein providing comprises administering SRRD or active fragment thereof to said subject.

18. The method of claim 16, wherein providing comprises administering an expression construct encoding SRRD or active fragment thereof to said subject.

19. The method of claim 18, wherein said expression construct is a viral expression construct, such as an adeno-associated virus construct.

20. The method of claim 18, wherein said expression construct is a non-viral expression construct.

21. The method of claims 16-20, wherein said SRRD or active fragment thereof further comprises a cell permeability peptide.

22. The method of claims 17-21, wherein said SRRD or active fragment thereof or expression construct encoding SSRD or active fragment thereof is administered in a liposome or nanoparticle.

23. The method of claims 16-22, wherein said subject is a mammal.

24. The method of claims 16-22, wherein said subject is a non-human mammal.

25. The method of claims 16-22, wherein said subject is a human.

26. The method of claims 16-25, wherein SRRD or active fragment thereof is provided to said subject more than once.

27. The method of claims 16-26, further comprising assessing protein aggregation in a cell of said subject prior to and/or after said providing.

28. The method of claims 16-27, further comprising administering to said subject a second anti-protein aggregation treatment.

29. The method of claims 16-28, wherein SRRD or active fragment thereof is provided at a level higher than normally expressed in a target cell in said subject.

30. The method of claims 16-29, wherein the protein aggregation disorder or disease is Alzheimer's Disease, Parkinson's Disease, amyotrophic lateral sclerosis, or frontotemporal dementia.

31. The method of claims 17-30, wherein SSRD or active fragment thereof or expression construct encoding SRRD or active fragment thereof is administered directly into the central nervous system, such as into the brain.

32. The method of claims 17-30, wherein SSRD or active fragment thereof or expression construct encoding SRRD or active fragment thereof is administered systemically, such as intravenously, intra-arterially, orally or subcutaneously.
